(19) United States
(12) Patent Application Publication
(10) Pub. No.: US 2023/0077514 A1
(43) Pub. Date: Mar. 16, 2023

(54) METHODS FOR MODULATING CALORIE CONSUMPTION

(71) Applicant: Duke University, Durham, NC (US)

(72) Inventor: Diego V. Bohórquez, Cary, NC (US)

(21) Appl. No.: 17/798,417

(22) PCT Filed: Feb. 17, 2021
(86) PCT No.: PCT/US2021/018325

§ 371 Date: Aug. 9, 2022

Related U.S. Application Data
(60) Provisional application No. 62/978,207, filed on Feb. 18, 2020.

(51) Int. Cl. A61K 31/675; A61K 31/47; A61K 31/662; A61K 45/06; A61B 5/00; A61B 5/16

(52) U.S. Cl. CPC A61K 31/675; A61B 5/165; A61B 5/4017; A61K 31/47; A61K 31/662; A61K 45/06

(57) ABSTRACT

Described herein are methods for modulating a transsynaptic signal through a neuroepithelial circuit between a gut sensory cell and the brain, as well as methods that involve modulating a transsynaptic signal through a neuroepithelial circuit between a gut sensory cell and the brain. In one aspect, the transsynaptic signal is modulated by glutamate or ATP.

### CROSS REFERENCE TO RELATED APPLICATIONS

This application claims priority to U.S. Provisional Pat. Application No. 62/978,207, filed on Feb. 18, 2020 which is incorporated by reference here in in its entirety.

### FEDERALLY SPONSORED RESEARCH

The subject matter of this invention was made with Government support under Federal Grant Nos. K01 DK-103832, DK 114500. The Government has certain rights to this invention.

### TECHNICAL FIELD

Described herein are methods for modulating a transsynaptic signal through a neuroepithelial circuit between a gut sensory cell and the brain, as well as methods that involve modulating a transsynaptic signal through a neuroepithelial circuit between a gut sensory cell and the brain. In one aspect, the transsynaptic signal is modulated by glutamate or ATP.

### BACKGROUND

Senses are used—sight, smell, texture, sound, and taste of food—to make choices about what to eat. Once ingested, food first hits the gut. There, cells in the villi of the small intestine function to both absorb nutrients and to communicate homeostatic and hedonic signals about what was eaten—to confirm or refute the information received by other senses. Berthoud et al., Gastroenterol. 152(7):1728-1738 (2017). The role of the gut in driving ingestive behavior was first studied as early as 1952. Miller and Kessen described the phenomenon that rats will work harder to attain a reward of infusion of milk into the stomach compared to an infusion of saline. Miller and Kessen, J. Compar. Physiol. Psych.45(6): 555-564 (1952). Since then, significant work has led to the notion that sugars infused directly into the gut are reinforcing. de Araujo et al., Neuron 57(6): 930-941 (2008); Beeler et al., Eur. J. Neurosci. 36(4): 2533-2546 (2012); Holman, J. Compar. Physiol. Psych. 69(3): 432-441 (1969); Sclafani and Ackroff, Physiol. Behav. 173: 188-199 (2017). Even in the absence of taste, hunger, or down-stream metabolism, caloric glucose infused into the duodenum elicits both a strong preference and dopamine release in the striatum. de Araujo et al., Neuron 57(6): 930-941 (2008); Ren et al., J. Neuroscience 30(23): 8012-8023 (2010); Sclafani etal., Amer. J. Phisol.: Reg. Integr. Compar. Physiol. 299(6): R1643-R1650 (2010); Zukerman et al., Amer. J. Phisol.: Reg. Integr. Compar. Physiol. 296(4): R866-R876 (2008); Yiin et al., Physiol. Behav. 84(2): 217-231 (2005); Zukerman et al., Amer. J. Phisol.: Reg. Integr. Compar. Physiol. 305(7): R840-R853 (2013); Tellez et al., Nature Neurosci. 19(3): 465-70 (2016).

In 1973, Gibbs et. al. noted that “within 10 min of starting to eat, a rat stops eating, grooms for a short period of time, then usually sleeps.” Gibbs et al., Nature 245: 323-325 (1973). For over thirty years, the field has focused on hormones as an explanation for this eating behavior, despite the fact that plasma CCK levels rise 10 or more minutes after nutrients enter the small intestine. Recently, Bellono et al. and Su et al. showed that AgRP hunger neurons in the hypothalamus are inhibited within seconds of sugar entering the small intestine. Bellono et al., Cell 170(1): 185-198 (2017); Su et al., Cell Rep. 21(10): 2724-2736 (2017). In 2015, we discovered that enteroendocrine cells synapse with nerves in the underlying intestinal and colonic mucosa. Bohorquez et al., J. Clin. Invest. 125(2): 782-786 (2015). In fact, when co-cultured with sensory neurons, they form a gut-brain neural circuit in a dish. Id. Such synapses have since been confirmed by other groups in enterochromaffin cells-a subset of gut epithelial sensors that secrete serotonin. Bellono et al., Cell 170(1): 185-198 (2017). In 2018, it was discovered that intestinal enteroendocrine cells synapse with vagal nodose neurons. These cells were called neuropod cells. They form a neural circuit that connects the intestinal lumen with the nucleus tractus solitarius in one synapse. This circuit is necessary and sufficient to transduce an intraluminal sucrose stimulus to the vagus nerve in as little as 60 milliseconds. See WO 2019/018438, which is incorporated by reference herein for such teachings.

What is needed is a method for modulating a transsynaptic signal through a neuroepithelial circuit between a gut sensory cell and the brain, as well as methods that involve modulating a transsynaptic signal through a neuroepithelial circuit between a gut sensory cell and the brain.

### SUMMARY

One embodiment described herein is a method for modulating a behavior or emotion through a neuroepithelial circuit between a gut sensory cell and the brain, the method comprising stimulating or inhibiting a transsynaptic signal from the gut sensory cell to the brain.

Another embodiment described herein is a method for modulating a behavior or emotion through a neuroepithelial circuit between a gut sensory cell and the brain, the method comprising stimulating or inhibiting glutamate release from the gut sensory cell.

Another embodiment described herein is a method for modulating a behavior or emotion through a neuroepithelial circuit between a gut sensory cell and the brain, the method comprising stimulating or inhibiting ATP release from the gut sensory cell. In one aspect, the behavior or emotion comprises feeding or consumption, hunger, satiety, appetite, craving, anxiety, depression, addiction, compulsion, pleasure, or combinations thereof. In another aspect, the transsynaptic signal is stimulated or inhibited by stimulating or inhibiting a receptor on the gut sensory cell. In another aspect, the receptor comprises a sweet taste receptor, a fatty acid receptor, a sodium glucose like transporter, or a combination thereof. In another aspect, the sweet taste receptor comprises a T1R2 subunit, a T1R3 subunit, or a combination thereof. In another aspect, the sodium glucose like transporter is SGLT1. In another aspect, the fatty acid receptor comprises Ffar2, Ffar3, Ffar4, Gpr119, Gp120, Cd36, or a combination thereof. In another aspect, a caloric sugar stimulates release of glutamate from the gut sensory cell. In another aspect, a non-caloric sugar stimulates release of ATP from the gut sensory cell. In another aspect, a caloric fatty acid stimulates release of glutamate from the gut sensory cell. In another aspect, a non-caloric fatty acid stimulates release of ATP from the gut sensory cell. In another aspect, the transsynaptic signal from the gut sensory cell to the brain is conveyed by the vagus nerve that comprises a glutamate receptor, an ATP receptor, or a combination thereof. In another aspect, stimulation of the glutamate receptor on the vagus nerve induces a preference for a caloric sugar in a subject. In another aspect, inhibition of the glutamate receptor on the vagus nerve abolishes the preference for a caloric sugar in a subject. In another aspect, stimulation of the glutamate receptor on the vagus nerve induces a preference for a caloric fatty acid in a subject. In another aspect, inhibition of the glutamate receptor on the vagus nerve abolishes the preference for a caloric fatty acid in a subject. In another aspect, stimulating or inhibiting the receptor on the vagus nerve comprises contacting the vagus nerve with a composition capable of stimulating or inhibiting the receptor on the vagus nerve. In another aspect, the composition comprises a modulator of the receptor. In another aspect, the modulator of the receptor comprises an agonist of the receptor, an antagonist of the receptor, or a combination thereof. In another aspect, the modulator of the receptor comprises a glutamate receptor antagonist. In another aspect, the glutamate receptor antagonist is kynurenic acid, D~/L~ 2-amino-3-phosphonopropionic acid (AP3), or a combination thereof. In another aspect, the modulator of the receptor comprises an ATP receptor antagonist. In another aspect, the ATP receptor antagonist is PPADS. In another aspect, a subject prefers a caloric sugar to a non-caloric sugar wherein inhibition of the gut sensory cell abolishes the preference. In another aspect, a subject prefers a caloric fatty acid to a non-caloric fatty acid wherein inhibition of the gut sensory cell abolishes the preference.

Another embodiment described herein is a method of modulating calorie consumption behavior or emotion through a neuroepithelial circuit between a gut sensory cell and the brain, the method comprising stimulating or inhibiting a transsynaptic signal from the gut sensory cell to the brain. In one aspect, the calorie consumption behavior or emotion in a subject is decreased. In another aspect, the calorie consumption behavior or emotion in a subject is increased. In another aspect, the calorie consumption behavior or emotion comprises feeding or consumption, hunger, satiety, appetite, craving, anxiety, depression, addiction, compulsion, pleasure, or combinations thereof.

Another embodiment described herein is a method for modulating a neurological or mental health condition comprising stimulating or inhibiting a transsynaptic signal from a gut sensory cell to the brain. In one aspect, the neurological or mental health conditions comprise anxiety, depression, autism, eating disorders, memory loss, neurologic pain, alcoholism, drug addiction, compulsive disorders, or combinations thereof. In another aspect, symptoms of the neurological or mental health condition are reduced. In another aspect, stimulating or inhibiting the receptor on the gut sensory cell comprises administering to a subject a therapeutically effective amount of a composition capable of stimulating or inhibiting the receptor on the gut sensory cell of the subject. In another aspect, stimulating or inhibiting the receptor on the gut sensory cell comprises contacting the gut sensory cell with a composition capable of stimulating or inhibiting the receptor on the gut sensory cell. In another aspect, the composition comprises a modulator of the receptor. In another aspect, the modulator of the receptor comprises an agonist of the receptor, an antagonist of the receptor, or a combination thereof. In another aspect, the modulator of the receptor comprises an SGLT1 antagonist.

Another embodiment described herein is a method for treating or ameliorating obesity comprising stimulating or inhibiting a transsynaptic signal from a gut sensory cell to the brain.

Another embodiment described herein is a method for distinguishing caloric from non-caloric sugars, the method comprising stimulating or inhibiting receptors on a gut sensory cell that modulates a transsynaptic signal from the gut sensory cell to the brain. In one aspect, the receptors comprise a sweet taste receptor, a sodium glucose like transporter, or a combination thereof. In another aspect, the sweet taste receptor comprises a T1R2 subunit, a T1R2R3 subunit, or a combination thereof. In another aspect, the sodium glucose like transporter is SGLT1. In another aspect, the caloric sugar comprises glucose, sucrose, maltodextrin, dextrose, maltose, fructose, galactose, or a combination thereof. In another aspect, the non-caloric sugar comprises sucralose, aspartame, saccharin, acesulfame-K, neotame, stevia, or a combination thereof. In another aspect, the composition comprises between about 0.5 mM and about 1000 mM of a caloric or non-caloric sugar. In another aspect, the composition comprises between about 2 mM and about 500 mM of a caloric or non-caloric sugar. In another aspect, the composition comprises at least about 100 mM a caloric or non-caloric sugar. In another aspect, the composition comprises at least about 300 mM sucrose. In another aspect, the composition comprises at least about 2 mM of sucralose. In another aspect, the transsynaptic signal from the gut sensory cell to the brain comprises a neuroepithelial circuit that comprises a nerve fiber. In another aspect, the neuroepithelial circuit comprises a gut sensory cell in contact with the nerve fiber. In another aspect, the gut sensory cell is in contact with the nerve fiber by releasing a neurotransmitter. In another aspect, the neurotransmitter is glutamate. In another aspect, the nerve fiber is a vagal nerve fiber or a sensory nerve fiber. In another aspect, the vagal nerve fiber includes a vagal nodose neuron. In another aspect, the gut sensory cell comprises a gut epithelial cell. In another aspect, the gut sensory cell comprises an enteroendocrine cell and/or an enterochromaffin cell. In another aspect, the neuroepithelial circuit comprises an enteroendocrine cell in contact with a vagal nerve fiber. In another aspect, the subject is a human subject. In another aspect, the receptor on the nerve fiber is an ionotropic glutamate receptor. In another aspect, the receptor on the nerve fiber is an N-methyl-D-aspartate (NMDA) receptor, an a-amino-3-hydroxy-5-methyl-4-isoxazolepropionic acid (AMPA) receptor, or a kainate receptor.

Another embodiment described herein is a method for distinguishing caloric from non-caloric fatty acids, the method comprising stimulating or inhibiting receptors on a gut sensory cell that modulates a transsynaptic signal from the gut sensory cell to the brain. In one aspect, the receptors comprise a fatty acid receptor. In another aspect, the fatty acid receptor comprises Ffar2, Ffar3, Ffar4, Gpr119, Gp120, Cd36, or a combination thereof.

### DESCRIPTION OF THE DRAWINGS

FIG. 1. Vagal responses to a variety of sugars depend on neuropod cells. Model: sugars are perfused into the duodenal lumen while nerve activity is recorded from the cervical vagus of anesthetized wild-type mice.

FIG. 2. The vagus responds significantly (p < 0.05) to whole (sucrose 300 mM, D-glucose 150 mM), calorie-only (maltodextrin 8% and α-mgp 150 mM), and non-caloric sugars (sucralose 15 mM, acesulfame K 15 mM) (n ≥ 5 wild-type mice per group). Fructose (150 mM) and saccharin (15 mM) responses are no significant (p < 0.05).

FIG. 3. Caloric and non-caloric sugars elicit a rapid response in vagal neurons. Intraluminal whole sugars (sucrose [300 mM], D-glucose [150 mM], fructose [150 mM], galactose [150 mM]), calorie-only sugars (alpha-methylglucopyranoside (a-MGP) [150 mM], and maltodextrin [8%]), and non-caloric sugars (sucralose [15 mM], acesulfame K (ace-K) [15 mM], and saccharin [15 mM]) intraluminally delivered while recording vagal activity. All sugars elicited significant vagal firing compared to baseline except for fructose and saccharin in wild-type mice (n ≥ 5 mice per group; *p < 0.001, ANOVA with post hoc Tukey’s HSD test).

FIG. 4. SGLT1 is used to sense caloric sugars and is expression consistently along the small intestine but is minimally in the colon. Caloric and non-caloric sugars act on different receptors (SGLT1 and T1R2R2/3) and are differentially metabolized (Met.).

FIG. 5. Minimal SGLT1 expression is visualized in the proximal colon. Scale bar is 100 µm.

FIG. 6. A cross-section of the entire length of the small intestine shows consistent SGLT1 (yellow) expression across the length (proximal = center). Scale bar is 1 mm.

FIG. 7. High concentrations of fructose (600 mM, 1 M) elicit a blunted vagal response (n ≥ 3 mice per group; statistics by ANOVA with post hoc Tukey’s HSD test). All shaded regions or error bars indicate SEM).

FIG. 8. In co-culture patch-clamp electrophysiology, the peak excitatory post-synaptic currents (EPSCs) elicited in connected nodose neurons was not significantly different between the application of D-glucose [20 mM] and sucralose [2 mM] (n = 18 pairs).

FIG. 9. Model: intraluminal 532 nm laser activates NpH3 chloride channels to silence duodenal Cck cells in CckCRE_NpH3 mice.

FIG. 10. Silencing duodenal Cck cells eliminates vagal responses to sucrose (300 mM), a-mgp (150 mM), and sucralose (15 mM).

FIG. 11. Quantified responses (n ≥ 5 mice per group; *p < 0.0001, ANOVA with post-hoc Tukey’s HSD test).

FIG. 12. Vagal nodose neurons cultured alone do not respond to D-glucose (20 mM), sucralose (2 mM), or maltodextrin (1%) (n = 59 neurons).

FIG. 13. Small intestinal CckCRE__tdTomato cells loaded with Fluo-4/FuraRed respond to D-glucose (20 mM), sucralose (2 mM), and maltodextrin (1%). Left-representative traces; Right-Venn diagram (n = 3 mice, n = 49 cells).

FIG. 14. Left-Patch-clamp electrophysiology of neurons in coculture with CckCRE__tdTomato cells (top-model; bottom-image, scale bar is 10 µm) Right-Of 18 co-culture pairs, excitatory post-synaptic potentials were recorded in neurons in response to D-glucose (20 mM, 44.4%), to sucralose only (2 mM, 22.2%), and to both stimuli (33.3%). Gray vertical bars indicate infusion period. Shaded regions on traces and error bars indicate SEM.

FIG. 15. Sweet taste receptors and the sodium glucose transporter 1 (SGLT1) are used by neuropod cells to distinguish caloric from non-caloric sugars. Single cell qRT-PCR of CckGFP cells and non GFP intestinal epithelial cells. Compared to nonGFP epithelial cells (n = 66), individual CckGFP cells (n = 132) express pre-synaptic genes Cplx1, Amigo1, Pclo, Syn1, Syn3, Syp, Snap25, Stxbp1 (N = 3 mice; q = 0.01).

FIG. 16. About 3 out of 4 CckGFP cells (green) in the proximal small intestine express SGLT1 (yellow) (n = 80-100 cells per mouse, N_EVref = 3 mice).

FIG. 17. Single cell qRT-PCR of CckGFP cells shows 19.6 ± 4.3% express detectable transcripts for both Slc5a1 (SGLT1) and Tas1r3 (T1R3), 60.1 ± 5.7% for only Slc5a1, 1.2 ± 1.2% for only Tas1r3, and 19.1 ± 1.2% for neither (n = 3 mice, n = 132 cells).

FIG. 18. Vagal nodose neurons do not express transcripts for SGLT1 or sweet taste receptors but do express transcripts for hormonal signaling and gut-brain glutamatergic and purinergic neurotransmission. Single cell transcriptomic data projected onto the vagal nodose atlas showing 18 Nodose (NG) and 6 Jugular (JG) clusters.

FIG. 19. Sensing transcripts for Slc5a1 (SGLT1), Tas1r2, and Tas1r3 are not expressed in vagal neurons.

FIG. 20. Model: neuropod cells express SGLT1 and T1R2R2/3, which are inhibited by phloridzin and gurmarin, respectively.

FIG. 21. Vagal responses to intra-duodenal sucrose (300 mM) and a-MGP (150 mM) are inhibited by SGLT1 inhibitor phloridzin (3 mM) and to sucralose (15 mM) by the T1R2/3 inhibitor gurmarin (7 µM).

FIG. 22. Quantification of data shown in FIG. 21; (*p < 0.0001, ANOVA with post-hoc Tukey’s HSD test). Shaded regions on traces or error bars indicate SEM. Scale bars, 10 µm

FIG. 23. Neuropod cells transmit signals from caloric and non-caloric sugars to the vagus using distinct neurotransmitters. Model: neuropod cells transduce caloric and non-caloric sugar signals to the vagus nerve using differential neurotransmitter release.

FIG. 24. Left-In CckGFP mouse intestinal organoids (CckGFP, green), sucrose (300 mM) and aMGP (150 mM) elicit significant release of glutamate compared to PBS control, while sucralose (15 mM) does not (n = 5-6 plates, N = 3 mice, *p < 0.05 by Student’s t-test). Right-Human duodenal organoids contain enteroendocrine cells (Chromogranin-A [ChgA], green) and release significant amounts of glutamate in response to sucrose (300 mM) and aMGP (150 mM) but no to sucralose (15 mM) or PBS control (n = 3-6 plates, N = 1 human sample, *p < 0.05 by Student’s t-test).

FIG. 25. Several NG clusters show expression of Cckar, Glutamatergic receptors, and ATP receptors (n = 5,507 cells; N_EVref = 5 right, and 6 left nodose ganglia).

FIG. 26. In wild-type mice, fast vagal responses to sucrose (300 mM) and the entire response to α-MGP (150 mM) are abolished by intraduodenal ionotropic/metabotropic glutamate receptor antagonists KA/AP3 [150 µg/kg]/[1 mg/kg]. Sucralose (15 mM) response is no affected by glutamate receptor antagonists.

FIG. 27. Murine neuropod cells use differential signaling molecules to distinguish caloric from non-caloric sugars. Glutamate receptor inhibition with KA/AP3 [150 pg/kg]/[1 mg/kg] prolonged time to peak vagal firing rate in response to sucrose [300 mM],

FIG. 28. Glutamate receptor inhibition reduced average area under the curve of vagal response to both sucrose [300 mM] and a-MGP [150 mM].

FIG. 29. Cck-A inhibition did not attenuate normalized maximum firing rate to sucrose [300 mM] or sucralose [15 mM].

FIG. 30. Cck-A inhibition did not significantly change time to peak vagal firing rate in response to sucrose [300 mM] or sucralose [15 mM].

FIG. 31. Cck-A inhibition reduced area under the curve of vagal response to sucrose [300 mM] but not sucralose [15 mM] compared to baseline (n ≥ 4 mice, *p < 0.05 ANOVA with post hoc Tukey’s HSD test. All shaded regions or error bars represent SEM.)

FIG. 32. Normalized traces for sucralose [15 mM] with and without the Cck-A receptor antagonist devazepide [1 mg/kg], and baseline post-inhibitor in wild type mice.

FIG. 33. Quantification of data shown in FIG. 32 (n = 5-11 mice, *p < 0.0001 ANOVA with post hoc Tukey’s HSD test).

FIG. 34. In wild-type mice, vagal responses to sucralose (15 mM) is abolished by the non-selective P2 purinoceptor antagonist PPADS [25 mg/kg]. Sucrose (300 mM) and a-MGP (150 mM) responses are not affected by the purinoceptor antagonist PPADS.

FIG. 35. Quantification of data shown in FIG. 34 (n = 6-7 mice, *p < 0.05 ANOVA with post hoc Tukey’s HSD test). Shaded regions or error bars indicate SEM. Scale bars are 10 µm.

FIG. 36. Non-selective P2 purinoceptor inhibition with PPADS [25 mg/kg] prolonged time to peak vagal firing rate to sucralose [15 mM].

FIG. 37. Non-selective P2 purinoceptor inhibition reduced area under the curve of vagal response to sucralose [15 mM].

FIG. 38. Duodenal neuropod cells drive choice of caloric over non-caloric sugars. Model: mice choose between sucrose (300 mM) or sucralose (15 mM) while consumption is recorded every 5 seconds.

FIG. 39. Within 2.08 minutes, wild-type mice choose sucrose over sucralose (n = 9 mice, *p < 0.05, repeated-measures ANOVA with post-hoc paired t-test).

FIG. 40. Systemic Cck receptor inhibition does not eliminate preference for sucrose. Mice are given a two-bottle preference test between sucrose [300 mM] and sucralose [15 mM] for 1 hour.

FIG. 41. Average traces showing sucrose [300 mM] and sucralose [15 mM] consumption with intraperitoneal injection 30 minutes prior to solution access of vehicle (PBS + 5% DMSO) in wild-type mice.

FIG. 42. Average traces showing sucrose [300 mM] and sucralose [15 mM] consumption with intraperitoneal injection 30 minutes prior to solution access of CCK-A receptor inhibitor devazepide ([2 mg/kg] in 10 µL/g mouse in 5% DMSO in PBS) in wild-type mice.

FIG. 43. Preference for sucrose over sucralose is unchanged by devazepide compared to vehicle in wild-type mice (n = 4, n.s. p = 0.6257 on repeated measures ANOVA). Total intake trends toward increasing with CCK-A receptor inhibition compared to vehicle control (n = 4, n.s. p = 0.2353 on repeated measures ANOVA).

FIG. 44. A flexible fiberoptic for optogenetic target of gut neuropod cells. Top-Schematic of the thermal drawing process for the flexible PC/PMMA waveguide fiber. A three-zone furnace applied controlled heat to the macroscopic template as the capstan pulls to reduce lateral dimensions by ~40 fold. Bottom-Cross section image of PC/PMMA core/cladding preform, as-drawn PC/PMMA flexible fiber, and photograph of -50 m fiber bundle.

FIG. 45. Light transmission for fibers bent at angles 90°, 180°, and 270° for varied radii of curvature. Plotted as percentage of output from a straight fiber.

FIG. 46. Light transmission loss through the length of fiber. Light transmission for straight and bent (angle = 180°, radius of curvature = 1 mm) flexible waveguides using the cut-back method. Percentage of light output from shortest length (Δ0 cm). Loss coefficients were calculated to be 0.93 dB/cm and 1.30 dB/cm for straight and bent fibers, respectively.

FIG. 47. Light transmission for flexible waveguides during cyclic bending at 180° with a 1 mm radius of curvature (odd cycles = straight, even = bent). Plotted as percentage of output from initial position (cycle = 0).

FIG. 48. Fiber flexibility was measured by a dynamic mechanical analyzer (DMA, Q800, TA Instrument) in a single cantilever mode with a displacement amplitude of 50 µm. Flexibility was measured for a standard silica fiber, and the PC/PMMA flexible fiber (for B-E, n = 3 fibers, shaded areas represent SD).

FIG. 49. Four weeks after implanting (intraduodenally), the flexible fiberoptic still illuminates.

FIG. 50. We developed a flexible fiberoptic for gut intraluminal optogenetics in awake and behaving mice. A conventional rigid fiber punctures an agar membrane, but the flexible fiber does not.

FIG. 51. Four weeks after implanting flexible fiberoptic in CckCRE_NpH3 mice, vagal recordings show its efficiency in silencing neuropod cells. Vertical bar indicates infusion period (n ≥ 4 mice; *p < 0.0001, ANOVA with post-hoc Tukey’s HSD test).

FIG. 52. Compared to 473 nm control laser, silencing duodenal neuropod cells with 532 nm laser in CckCRE_NpH3 mice eliminates sucrose preference (n = 8).

FIG. 53. Preference quantified at one hour with no laser (pre/post), 473 nm laser, and 532 nm laser (n = 8).

FIG. 54. Silencing neuropod cells with 532 nm laser in CckCRE_NpH3 mice decreases sucrose and increases sucralose consumption but does affect total consumption (n = 8).

FIG. 55. In wild-type mice, intraduodenal perfusion of KA/AP3 [5 ng/0.1 µg in 0.4 mL over 1 hour] to inhibit glutamate receptors eliminates sucrose preference (n = 4).

FIG. 56. Sucrose preference elimination is due to effect of laser inhibition on neuropod cells in CckCRE_Halorhodopsin mice. While the laser was pulsed at 40 Hz, 1-minute ON 2-minutes OFF, preference for sucrose over sucralose was significantly reduced both during periods of laser on and periods of laser off in Cck_Halo mice (n = 8, *p < 0.01 by repeated measures ANOVA with paired t-test post-hoc analysis).

FIG. 57. Activity is unchanged by 473 nm (control) and 532 nm (silencing) in Cck_Halo mice (n ≥ 7, n.s. (p > 0.05) by repeated measures ANOVA).

FIG. 58. 532 nm silencing laser did not affect 24-hour intake of chow or water compared to 473 nm control laser (n ≥ 6, *p < 0.01 by repeated measures ANOVA with paired t-test post-hoc analysis).

FIG. 59. Average traces showing sucrose [300 mM] and sucralose [15 mM] consumption during 473 nm (control, left) laser and 532 nm (silencing, right) laser inhibition in genotype-negative littermate controls of CckCRE_NpH3 mice.

FIG. 60. Preference for sucrose over sucralose is unchanged by 532 nm or 473 nm laser in genotype-negative littermate control mice.

FIG. 61. 532 nm or 473 nm does not affect overall intake of sucrose, sucralose, and total intake in control mice (n = 5 littermate controls; n.s. (p > 0.05) by repeated measures ANOVA).

FIG. 62. Systemic glutamate receptor inhibition recapitulates preference elimination. Mice are given a two-bottle preference test between sucrose [300 mM] and sucralose [15 mM] for 1 hour.

FIG. 63. Average traces showing sucrose [300 mM] and sucralose [15 mM] consumption with intraperitoneal injection 5 minutes prior to solution access of vehicle (PBS + 1 M NaOH, pH 7.4) in wild-type mice.

FIG. 64. Average traces showing sucrose [300 mM] and sucralose [15 mM] consumption with intraperitoneal injection 5 minutes prior to solution access of ionotropic/metabotropic glutamate receptor inhibitors kynurenic acid (KA)/AP-3 ([150 µg/kg]/[1 mg/kg] in 10 µL/g mouse in PBS pH 7.4 from 1 M NaOH stocks) in wild-type mice.

FIG. 65. Preference for sucrose over sucralose is significantly attenuated by systemic glutamate receptor inhibition compared to vehicle (n = 4, * p < 0.05 on repeated measures ANOVA with post-hoc paired t-test).

FIG. 66. Preference quantified at 1 hour with PBS (pre/post), vehicle, and glutamate receptor blockers KA/AP3 (n = 4).

FIG. 67. Inhibiting glutamate receptors increases sucralose consumption without affecting sucrose or total consumption (n = 4; *p < 0.05, repeated-measures ANOVA with post-hoc paired t-test). Shaded regions or error bars indicate SEM.

FIG. 68. Vagal firing rate is increased by duodenal long-chain fatty acids (n = 9; p < 0.005).

FIG. 69. Cck gut epithelial cells express fatty acid receptors (Ffar2, Ffar3, Ffar4, Gpr1 19).

FIG. 70. Cck gut epithelial cells are required for fat-elicited vagal activity (n = 3; p < 0.05).

FIG. 71. Glutamate signaling is required for fat-elicited vagal activity (n = 5, wild type).

FIG. 72. Intralipid, but not sucrose, elicits real time place preference (n = 6, wild type).

FIG. 73. Intralipid real time place preference requires glutamate signaling (n = 3, wild type).

FIG. 74. Intralipid real time place preference requires glutamate signaling (n = 2, CckCRE_ChR2).

### DETAILED DESCRIPTION

Animals, including humans, have an innate ability to prefer caloric sugars (e.g. sucrose) over non-caloric sugars (e.g. sucralose). The signals arise in the intestine. Previous research discovered an intestinal cell type that synapses with the vagus nerve, which are termed “neuropod cells.” The connection that neuropod cells form with the vagus nerve is sufficient and necessary to pass signals from sucrose up to the brain in seconds. Described herein is a mechanism through which neuropod cells drive the preference for caloric sugars in a mouse model. The mechanism involves the activation of the sodium glucose transporter 1 in neuropod cells and the subsequent release of the neurotransmitter glutamate from neuropod cells. Described herein is the discovery that glutamate release by neuropod cells is necessary for the animal mouse model to choose sucrose over sucralose. Inhibiting the entire neuropod cell, its sodium glucose transporter 1 channels, or the vagal receptors for glutamate impairs the animal’s ability to detect caloric sugars. In human intestinal organoids, which contain neuropod cells, a stimulus sucrose also causes the release of glutamate, demonstrating that the mechanism is conserved in humans. This mechanism is a target for drug-based therapies, microbial approaches, or nutraceuticals to alter the consumption of caloric sugars and reduce body weight gain.

The present disclosure is based, in part, on the discovery of the components and functional connectivity of the gut neuroepithelial circuit; a neural circuit for fast transduction of gut-brain signals. It was previously discovered that enteroendocrine cells synapse with the vagus to transduce sensory signals from the gut to the brain. This gut-to-brain sensory neuroepithelial circuit can act as a portal for pathogens and a path for luminal stimuli, such as ingested sugars, to reach the brain. Enteroendocrine cells are constantly exposed to luminal contents of the small intestine and colon. Through the expression of cell membrane receptors, they recognize nutrients, bacterial ligands, mechanical stretch, and perhaps thermal signals in the lumen of the small intestine and colon. The connection to neurons indicates that nerves can sense luminal contents through rapid neuronal excitation and pathogens that infect enteroendocrine cells can spread to the nervous system through direct synaptic links. A monosynaptic link between gut sensory cells, such as gut sensory epithelial cells, and nerves, such as vagal nodose neurons, was surprisingly discovered and observed both in vivo and in vitro. This neural circuit can constitute a path for pathogens in the gut to travel up to the brain, including viruses that cause neurodegenerative diseases. This claim is clearly shown by the rabies discovery and the ability to recapitulate the connection with organoids. Another discovery was that enteroendocrine cell, such as CCK enteroendocrine cells, produced signaling molecules that were previously ascribed only to enterochromaffin cells. The discovered neuroepithelial circuit was capable of transducing signals from nutrients, such as glucose, within milliseconds (60-800 ms), opening a physical path for fast sensory transduction from gut lumen to brain, analogous to that of taste transduction in the tongue. For example, the enteroendocrine cells transduced sensory signals from gut lumen, including that of glucose, within milliseconds to vagal nodose neurons.

A monosynaptic rabies virus and optogenetics was previously used to dissect details of this sensory transduction from the gut to the brain. See WO 2019/018438, the contents of which are fully incorporated by reference herein. To define if peripheral nerves connect with enteroendocrine cells, the monosynaptic rabies virus B19G SADΔG-GFP was used with a transgenic mouse model, PyyCRE_tdTomato_rabG, that was developed to enable cell-specific spread of the virus. When delivered in the colon’s lumen, there was visible GFP in mucosal nerves and nodose of PyyCRE_tdTomato_rabG mice but not in controls. These data show that colonic enteroendocrine cells are innervated by vagal nerve fibers. To test neurotransmission in this neuroepithelial circuit in isolation, an in vitro co-culture system was developed using purified enteroendocrine cells and nodose ganglia neurons. The two cell types connected within 12-36 hrs and the connected cells often remained viable for at least 5 days, showing that this neuroepithelial circuit could be recapitulated in vitro. The possibility of afferent gut-to-brain transduction using whole cell electrophysiology was tested and it was discovered that a stimulus of 10 mM of glucose applied to the enteroendocrine cell induced excitatory post-synaptic potentials and action potential spikes in the connected neuron, while 10 mM glucose did not activate a nodose neuron by itself. These findings unveiled a gut-brain neuroepithelial circuit with the ability to transduce a chemical sense.

By synapsing with the vagus, these sensors provide a neuroepithelial circuit for fast sensory transduction. Gut sensory epithelial cells that synapse with nerves are called neuropod cells. The gut brain neural circuit can be formed by neuropod cells and nodose neurons. The existence of this neural circuit leads to: (1) rapid distinction of stimuli based on physical (e.g. volume) versus chemical (e.g. calorie) composition; (2) precise topographical sensory representation of specific gastrointestinal regions; (3) localized plasticity encoded within the neural circuit, depending on the stimuli; (4) timely vagal efferent feedback to modulate gastrointestinal sensory function; and (5) given the conditions, a portal for gut-borne pathogens to gain access to the central nervous system. Enteroendocrine cells can use both, paracrine and neurotransmission signals, to help the brain make sense of what is being eaten by a subject.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Section headings as used in this section and the entire disclosure herein are merely for organizational purposes and are not intended to be limiting.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments "comprising," "consisting of and "consisting essentially of," the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Enterochromaffin cell” as used herein refers to a type of enteroendocrine and neuroendocrine cell that resides alongside the epithelium lining of the lumen of the digestive tract. Enterochromaffin cells, also known as Kulchitsky cells, play a role in gastrointestinal regulation, particularly intestinal motility, and secretion. Enterochromaffin cells modulate neuron signaling in the enteric nervous system (ENS) via the secretion of the neurotransmitter serotonin and other peptides. Enterochromaffin cells act as a form of sensory transduction as enteric afferent and efferent nerves do not protrude into the intestinal lumen.

“Enteroendocrine cells” as used herein refers to specialized cells of the gastrointestinal tract and pancreas with endocrine function. Enteroendocrine cells produce gastrointestinal hormones or peptides in response to various stimuli and release them into the bloodstream for systemic effect, diffuse them as local messengers, or transmit them to the enteric nervous system to activate nervous responses. Enteroendocrine cell constitute an enteric endocrine system as a subset of the endocrine system and are known to act as chemoreceptors, initiating digestive actions and detecting harmful substances and initiating protective responses. Enteroendocrine cells are located in the stomach, in the intestine and in the pancreas. Enteroendocrine cells can be intestinal enteroendocrine cells, gastric enteroendocrine cells, or pancreatic enteroendocrine cells, including but limited to, K cell, I cell, I cell, G cell, enterochromaffin cell, N cell, S cell, D cell, and M cell.

The terms “inhibitor”, “antagonist” or “blocker” refer to a compound or substance that decreases or blocks one or more activities of a protein of interest, for example, a sugar-sensing receptor, an amino acid-sensing receptor, a fatty acid-sensing receptor, or a bacteria-sensing receptor. In specific embodiments, terms “inhibitor,” “antagonist,” or “blocker” in the context of the receptors, refer to a compound or substance that decreases the downstream signaling response associated with the receptor. In particular embodiments, decreasing receptor activity can result in change in the amount or distribution of an intracellular molecule or the activity of an enzyme which is part of the intracellular signaling pathway for the receptor. Examples of the intracellular molecule include, but are not limited to, free calcium, cyclic adenosine monophosphate (cAMP), inositol mono-, di-, or tri-phosphates. Examples of the enzyme include, but are not limited to, adenylate cyclase, phospholipase-C, and G-protein coupled receptor kinases.

“Subject” and “patient” as used herein interchangeably refers to any vertebrate, including, but not limited to, a mammal and a human. In some embodiments, the subject may be a human or a non-human. The subject or patient may be undergoing forms of treatment. “Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

“Treat,” “treating” or “treatment” are each used interchangeably herein to describe reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

Unless otherwise defined herein, scientific, and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### Methods of Modulating a Behavior or Emotion Through a Neuroepithelial Circuit Between a Gut Sensory Cell and the Brain

Embodiments of the present disclosure relate generally to a method of modulating a behavior or emotion through a neuroepithelial circuit between a gut sensory cell and the brain. The method includes stimulating or inhibiting a transsynaptic signal from the gut sensory cell to the brain. In some embodiments, stimulating or inhibiting the receptor on the gut sensory cell comprises contacting the gut sensory cell with a composition capable of stimulating or inhibiting the receptor on the gut sensory cell. In some embodiments, stimulating or inhibiting the receptor on the gut sensory cell comprises administering to the subject a therapeutically effective amount of a composition capable of stimulating or inhibiting the receptor on the gut sensory cell of the subject. In some embodiments, the method may include stimulating or inhibiting glutamate release from the gut sensory cell. In some embodiments, the method may include stimulating or inhibiting ATP release from the gut sensory cell. In some embodiments, stimulating or inhibiting the glutamate and/or ATP release from the gut sensory cell comprises contacting the gut sensory cell with a composition capable of stimulating or inhibiting the glutamate and/or ATP release from the gut sensory cell. In some embodiments, stimulating or inhibiting the glutamate and/or ATP release from the gut sensory cell comprises administering to the subject a therapeutically effective amount of a composition capable of stimulating or inhibiting the glutamate and/or ATP release from the gut sensory cell of the subject. In some embodiments, the composition includes a modulator of the receptor. In some embodiments, the receptor can be involved in sensing a carbohydrate, such as a caloric sugar, a starch, or a cellulose, an amino acid, a protein, a fatty acid, a fat, or a bacterium or bacteria. In some embodiments, the receptor can be involved in sensing a non-caloric sugar. In some embodiments, the caloric sugar may be glucose, sucrose, maltodextrin, dextrose, maltose, fructose, or galactose. In some embodiments, the non-caloric sugar may be sucralose, aspartame, saccharin, acesulfame-K, neotame, or stevia. In some embodiments, the receptor can be a sugar-sensing receptor, an amino acid-sensing receptor, a fatty acid-sensing receptor, or a bacteria-sensing receptor.

A “sweet taste receptor” as used herein refers to receptor that binds and recognizes a caloric or non-caloric sugar. In some embodiments, the sweet taste receptor can consist of a taste receptor type 1 (T1R) subunit, such as T1R2 and/or T1R3. T1R2 and T1R3 are found in enteroendocrine cells of the gastrointestinal tract. T1R2 which is encoded by the TAS1R2 gene and T1R3 which is encoded by the TAS1R3 gene, are G protein-coupled receptors with seven trans-membrane domains and are a component of the heterodimeric amino acid taste receptor T1R2+3.

A sodium-dependent glucose cotransporter or sodium-glucose linked transporter (SGLT) as used herein refers to a receptor that binds and recognizes a caloric sugar, such as glucose. SGLT1, which is a part of the family of glucose transporters, is found in the intestinal mucosa of the small intestine.

An “amino acid sensing receptor” or “amino acid receptor” as used herein refers to a receptor that binds and recognizes an amino acid. In some embodiments, the amino acid can be an L-amino acid, such as L-glutamate, or a D-amino acid, such as D-glutamate. In some embodiments, the amino acid-sensing receptor can be a taste receptor type 1 member 1 (TAS1R1). TAS1R1, which is encoded by the TAS1R1 gene, is a G protein-coupled receptor with seven trans-membrane domains and is a component of the heterodimeric amino acid taste receptor T1R1+3. In some embodiments, the amino acid-sensing receptor can be a vesicular glutamate transporter, such as Vglut1.

A “fatty acid-sensing receptor” or “fatty acid receptor” as used herein refers to a receptor that binds and recognizes a fatty acid. In some embodiments, the fatty acid can be a short chain saturated fatty acid, a short chain unsaturated fatty acid, a medium chain saturated fatty acid, a medium chain unsaturated fatty acid, a long chain saturated fatty acid, or a long chain unsaturated fatty acid. In some embodiments the fatty acid can be eicosatrienoic acid (20:3Δ11,14,17), linoleic acid, oleic acid, or have at least 10 carbons. In some embodiments, the fatty acid-sensing receptor can be free fatty acid receptor 1 (FFA1), also known as GPR40, free fatty acid receptor 2 (FFA3), free fatty acid receptor 3 (FFA3), free fatty acid receptor 4 (FFA4, also known as G protein-coupled receptor 120 (GPR120)), or G protein-coupled receptor 119 (GPR119).

A “bacteria-sensing receptor” as used herein refers to a receptor that binds and recognizes structurally conserved molecules derived from microbes. In some embodiments, the molecules derived from microbes can be a bacterial lipoprotein, bacterial glycolipids, bacterial lipopolysaccharide, bacterial lipoteichoic acid, fungal zymosan (P-glucan), viral double-stranded RNA, poly I:C, bacterial heat shock proteins, peptidoglycan motifs from bacterial cell which consists of N-acetylglucosamine and N-acetylmuramic acid, such as meso-diaminopimelic acid (meso-DAP) or muramyl dipeptide (MDP). In some embodiments, the bacterial-sensing receptor can be Toll-like receptor 5 (TLR5), Toll-like receptor 1 (TLR1), Toll-like receptor 2 (TLR2), Toll-like receptor 3 (TLR3), Toll-like receptor 4 (TLR4), Tlr1, Tlr2, Tlr3, Tlr4, Tlr5, nucleotide-binding oligomerization domain-containing protein 1 (NOD1), or nucleotide-binding oligomerization domain-containing protein 2 (NOD2).

In some embodiments, the receptor can be a sodium-dependent glucose cotransporter (SGLT), a taste receptor type receptor (TAS), a free fatty acid receptor (FFAR), a G-protein coupled receptor (GPR), a Toll-like receptor (TLR), a nucleotide-binding oligomerization domain-containing protein receptor (NOD), or a combination thereof. In some embodiments, the receptor can be SGLT1 or TLR5.

In some embodiments, the neuroepithelial circuit can include a nerve fiber. In some embodiments, the neuroepithelial circuit can include a gut sensory cell in contact with the nerve fiber. In some embodiments, the gut sensory cell is in contact or communication with the nerve fiber by releasing a neurotransmitter. In some embodiments, the neurotransmitter is glutamate. In some embodiments, the nerve fiber is a vagal nerve fiber or a sensory nerve fiber. In some embodiments, the vagal nerve fiber can include a vagal nodose neuron. In some embodiments, the gut sensory cell can include a gut epithelial cell. In some embodiments, the gut sensory cell can include an enteroendocrine cell and/or an enterochromaffin cell. By synapsing with vagal neurons, enteroendocrine cells can transduce signals up to the brain in a direct, selective, and temporally precise fashion. In some embodiments, the enteroendocrine cell is from the small intestine or colon. In some embodiments, the neuroepithelial circuit can include an enteroendocrine cell in contact with a vagal nerve fiber.

In some embodiments, the neuroepithelial circuit can be stimulated or inhibited by modulating the communication of the gut sensory cell with the nerve fiber. In some embodiments, modulating the communication of the gut sensory cell can include administering an inhibitor of the neurotransmitter. In some embodiments, the inhibitor of the neurotransmitter blocks the neurotransmitter from binding to the nerve fiber. In some embodiments, the neurotransmitter is glutamate and the inhibitor blocks an ionotropic glutamate receptor on the nerve fiber. In some embodiments, the ionotropic glutamate receptor is an N-methyl-D-aspartate (NMDA) receptor, an α-amino-3-hydroxy-5-methyl-4-isoxazolepropionic acid (AMPA) receptor or a kainate receptor. In some embodiments, the ionotropic glutamate receptor inhibitor is kynurenic acid or D-/L-2-amino-3-phosphonopropionic acid (AP-3). In some embodiments, the neurotransmitter is adenosine triphosphate (ATP) and the inhibitor blocks an ATP-gated P2X receptor cation channel, such as P2X₁, P2X₂, P2X₃, or P2X₅. In some embodiments, the P2X receptor is inhibited with pyridoxalphosphate-6-azophenyl-2’,4'-disulfonic acid (PPADS).

### Modulators of the Receptor

In some embodiments, stimulating or inhibiting a receptor on the gut sensory cell can include contacting the gut sensory cell with a composition capable of stimulating or inhibiting the receptor on the gut sensory cell. In some embodiments, stimulating or inhibiting a receptor on the gut sensory cell can include administering to the subject a therapeutically effective amount of a composition capable of stimulating or inhibiting the receptor on the gut sensory cell of the subject. In some embodiments, the composition can include a modulator of the receptor.

In some embodiments, the modulator of the receptor can include an agonist of the receptor, an antagonist of the receptor, and/or an inhibitor of the receptor.

In some embodiments, the modulation is achieved using a modulator of the sweet taste receptor. In some embodiments, the modulator can be an agonist, antagonist, or inhibitor of the sugar-sensing receptor. For example, the modulator can be an T1R2/3 inhibitor, such as gurmarin. In some embodiments, the modulation is achieved using a modulator of the SGLT receptor. In some embodiments, the modulator can be an agonist, antagonist, or inhibitor of the SGLT receptor. For example, the modulator can be an SGLT1 inhibitor, such as GSK1614235, phloridzin, phloridzin dihydrate, sotagliflozin, DSP-3235, or T-1095. In some embodiments, the modulator of the receptor can include an SGLT1 inhibitor. In some embodiments, the SGLT1 inhibitor can be phloridzin dehydrate.

In some embodiments, the composition can include a caloric sugar or a non-caloric sugar. In some embodiments, the composition can include a caloric sugar, such as glucose, sucrose, maltodextrin, dextrose, maltose, fructose, galactose, or a combination thereof. The composition can include a non-metabolizable sugar, such as α-methyl-D-glucopyranoside (αMG). In some embodiments, the non-caloric sugar can include sucralose, aspartame, saccharin, acesulfame-K, neotame, stevia, or a combination thereof.

In some embodiments, the composition can include between about 0.5 mM and about 1000 mM of sugar. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 1000 mM of sugar. In some embodiments, the composition can include between about 10 mM and about 500 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of sugar. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of sugar. In some embodiments, the composition can include at least about 100 mM sugar.

In some embodiments, the composition can include between about 0.5 mM and about 1000 mM of glucose. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of glucose. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of glucose. In some embodiments, the composition can include at least about 300 mM to about 800 mM glucose.

In some embodiments, the composition can include between about 0.5 mM and about 1000 mM of sucrose. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM., between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of sucrose. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of sucrose.

In some embodiments, the composition can include between about 0.5 mM and about 1000 mM of sucralose. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM., between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of sucralose. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of sucralose. In some embodiments, the composition can include at least about 15 mM of sucralose.

In some embodiments, the composition can include between about 0.5 mM and about 1000 mM of fructose. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 400 mM, between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of fructose. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of fructose.

In some embodiments, the composition can include between about 0.5 mM and about 1000 mM of maltodextrin. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 400 mM, between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of maltodextrin. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of maltodextrin.

In some embodiments, the composition can include between about 0.5 mM and about 1000 mM of dextrose. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 400 mM, between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of dextrose. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of dextrose.

In some embodiments, the composition can include between about 0.5 mM and about 1000 mM of maltose. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 400 mM, between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of maltose. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of maltose.

In some embodiments, the composition can include between about 0.5 mM and about 1000 mM of galactose. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 400 mM, between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of galactose. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of galactose.

In some embodiments, the composition can include between about 0.5 mM and about 1000 mM of aspartame. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 400 mM, between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of aspartame. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of aspartame.

In some embodiments, the composition can include between about 0.5 mM and about 1000 mM of saccharin. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 400 mM, between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of saccharin. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of saccharin.

In some embodiments, the composition can include between about 0.5 mM and about 1000 mM of acesulfame-K. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 400 mM, between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of acesulfame-K. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of acesulfame-K.

In some embodiments, the composition can include between about 0.5 mM and about 1000 mM of neotame. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 400 mM, between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of neotame. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of neotame.

In some embodiments, the composition can include between about 0.5 mM and about 1000 mM of stevia. In some embodiments, the composition can include between about 0.5 mM and about 500 mM, between about 0.5 mM and about 250 mM, between about 0.5 mM and about 200 mM, between about 0.5 mM and about 150 mM, between about 0.5 mM and about 100 mM, between about 0.5 mM and about 50 mM, between about 1 mM and about 500 mM, between about 1 mM and about 250 mM, between about 1 mM and about 200 mM, between about 1 mM and about 150 mM, between about 1 mM and about 100 mM, between about 1 mM and about 50 mM, between about 5 mM and about 500 mM, between about 5 mM and about 250 mM, between about 5 mM and about 200 mM, between about 5 mM and about 150 mM, between about 5 mM and about 100 mM, between about 5 mM and about 50 mM, between about 10 mM and about 500 mM, between about 10 mM and about 400 mM, between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of stevia. In some embodiments, the composition can include at least about 0.5 mM, at least about 1 mM, at least about 5 mM, at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM, at least about 500 mM, at least about 525 mM, at least about 550 mM, at least about 575 mM, at least about 600 mM, at least about 625 mM, at least about 650 mM, at least about 675 mM, at least about 700 mM, at least about 725 mM, at least about 750 mM, at least about 775 mM, at least about 800 mM, at least about 825 mM, at least about 850 mM, at least about 875 mM, at least about 900 mM, at least about 925 mM, at least about 950 mM, at least about 975 mM, or at least about 1000 mM of stevia.

### Methods of Modulating Calorie Consumption Behavior or Emotion of a Subject

The present disclosure also relates to methods of modulating calorie consumption behavior or emotion of a subject through a neuroepithelial circuit between a gut sensory cell and the brain. The method includes stimulating or inhibiting a transsynaptic signal from the gut sensory cell to the brain and modulating the calorie sensing in the subject. By these the methods, the subject modulates the consumption behavior or emotion of the subject, such as modulating feeding/consumption, hunger, satiety, craving, anxiety, depression, addiction, compulsion, pleasure, appetite, food intake, and/or food preference, in a manner that is different than if the receptor was not stimulated or inhibited in the subject. In some embodiments, stimulating the transsynaptic signal from the gut sensory cell to the brain increases the calorie consumption behavior and/or emotion of the subject. In some embodiments, stimulating the transsynaptic signal from the gut sensory cell to the brain decreases the calorie consumption behavior and/or emotion of the subject.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described herein. In some embodiments, the composition includes a modulator of the receptor, as described herein. In some embodiments, the receptor can be involved in sensing a caloric sugar or non-caloric sugar, as described herein. In some embodiments, the receptor can be a sweet taste receptor or a SGLT receptor, as described herein.

### Methods of Modulating Food Intake Behavior in a Subject

The present disclosure also provides methods of modulating food intake behavior in a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the food intake in a subject is modulated. In some embodiments, the food intake behavior in the subject is increased. In some embodiments, the food intake behavior in the subject is decreased.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described herein. In some embodiments, the composition includes a modulator of the receptor, as described herein. In some embodiments, the receptor can be involved in sensing a caloric sugar or non-caloric sugar, as described herein. In some embodiments, the receptor can be a sweet taste receptor or a SGLT receptor as described herein.

### Methods of Modulating Food Preference in a Subject

The present disclosure also provides methods of modulating food preference in a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the food preference behavior in the subject is modulated. In some embodiments, the food intake behavior and/or food preference in the subject is increased. In some embodiments, the food intake behavior and/or food preference in the subject is decreased.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described herein. In some embodiments, the composition includes a modulator of the receptor, as described herein. In some embodiments, the receptor can be involved in sensing a caloric sugar or non-caloric sugar, as described herein. In some embodiments, the receptor can be a sweet taste receptor or a SGLT receptor, as described herein.

### Methods of Treating a Subject Having or Suspected of Having a Neurological and/or Mental Health Condition

The present disclosure also provides methods of treating a subject having or suspected of having a neurological and/or mental health condition. The method involves modulating brain function through the GI tract to treat neurological and/or mental health disorders that have both neurological and gastrointestinal components, such as anxiety, depression, autism, eating disorders (e.g. anorexia and bulimia), memory loss, neurologic pain, alcoholism, drug addiction, compulsive disorders, or combinations thereof. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein eating behavior of the subject is modulated and the subject is treated. In some embodiments, the eating behavior of the subject is increased. In some embodiments, the eating behavior of the subject is decreased.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described herein. In some embodiments, the composition includes a modulator of the receptor, as described herein. In some embodiments, the receptor can be involved in sensing a caloric sugar or non-caloric sugar, as described herein. In some embodiments, the receptor can be a sweet taste receptor or SGLT receptor as described herein.

### Methods of Treating or Ameliorating Obesity in a Subject

The present disclosure also provides methods of treating or ameliorating obesity in a subject. The method involves modulating calorie consumption through stimulating or inhibiting a transsynaptic signal from a gut sensory cell to the brain. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein eating behavior of the subject is modulated and the subject is treated. In some embodiments, the eating behavior of the subject is increased. In some embodiments, the eating behavior of the subject is decreased.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described herein. In some embodiments, the composition includes a modulator of the receptor, as described herein. In some embodiments, the receptor can be involved in sensing a caloric sugar or non-caloric sugar, as described herein. In some embodiments, the receptor can be a sweet taste receptor or SGLT receptor as described herein.

### Methods of Distinguishing a Caloric Sugar From a Non-Caloric Sugar

The present disclosure provides methods of distinguishing a caloric sugar from a non-caloric sugar. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the food preference behavior in the subject is modulated. In some embodiments, the caloric sugar stimulates release of glutamate from the gut sensory cell. In some embodiments, the glutamate stimulates a glutamate receptor on a nerve, such as the vagus nerve or a sensory nerve. In some embodiments, the glutamate receptor may be an ionotropic glutamate receptor, such as AMPA, NMDA, delta, or kainite receptors. In some embodiments, the non-caloric sugar stimulates release of ATP from the gut sensory cell. In some embodiments, the ATP stimulates an ATP receptor on a nerve, such as the vagus nerve or a sensory nerve. In some embodiments, the ATP receptor may be an ATP-gated P2X receptor cation channel, such as P2X₁, P2X₂, P2X₃, or P2X₅. In some embodiments, the food intake behavior and/or food preference in the subject is increased. In some embodiments, the food intake behavior and/or food preference in the subject is decreased.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described herein. In some embodiments, the composition includes a modulator of the receptor, as described herein. In some embodiments, the receptor can be involved in sensing a caloric sugar or non-caloric sugar, as described herein. In some embodiments, the receptor can be a sweet taste receptor or a SGLT receptor, as described herein.

### Compositions, Pharmaceutical Compositions, and Formulations

Embodiments of the present disclosure also provide compositions, pharmaceutical compositions, and formulations that include at least one modulator of the receptor. The disclosed compositions, pharmaceutical compositions, and formulations can be used to treat or alleviate the symptoms of subjects that are diagnosed with or determined as having a neurological and/or mental health condition. The disclosed compositions, pharmaceutical compositions, and formulations can include at least one modulator of the receptor.

The compositions, pharmaceutical compositions, and formulations may include a “therapeutically effective amount” or a “prophylactically effective amount” of the modulator of the receptor. A “therapeutically effective amount” refers to an amount effective, at dosages and for periods of time necessary, to achieve the desired therapeutic result. A therapeutically effective amount of the compositions may be determined by a person skilled in the art and may vary according to factors such as the disease state, age, sex, and weight of the individual, and the ability of the compositions to elicit a desired response in the individual. A therapeutically effective amount is also one in which any toxic or detrimental effects of the modulator of the receptor, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of the modulator of the receptor, calculated to produce the desired therapeutic effect in association with the required pharmaceutical carrier. The specification for the dosage unit forms are dictated by and directly dependent on (a) the unique characteristics of modulator of the receptor, and the particular therapeutic or prophylactic effect to be achieved, and (b) the limitations inherent in the art of compounding such composition that modulates the receptor.

It is to be noted that dosage values may vary with the type and severity of the condition to be alleviated. Further, the modulator of the receptor dose may be determined by a person skilled in the art and may vary according to factors such as the disease state, age, sex, and weight of the individual, and the ability of the modulator of the receptor to elicit a desired response in the individual. The dose is also one in which toxic or detrimental effects, if any, of the modulator of the receptor are outweighed by the therapeutically beneficial effects. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The compositions, pharmaceutical compositions, and formulations may include pharmaceutically acceptable carriers. The term “pharmaceutically acceptable carrier,” as used herein, means a non-toxic, inert solid, semi-solid or liquid filler, diluent, encapsulating material, or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; water; isotonic saline; Ringer’s solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Various delivery systems are known and can be used to administer one or more of modulator of the receptor, and a prophylactic agent or therapeutic agent useful for preventing, managing, treating, or ameliorating the neurological and/or mental health condition as described herein, or one or more symptoms thereof, e.g., encapsulation in liposomes, microparticles, microcapsules. Methods of administering a prophylactic or therapeutic agent of the invention include, but are not limited to, parenteral administration (e.g., intradermal, intramuscular, intraperitoneal, intravenous, or subcutaneous), epidural administration, intratumoral administration, and mucosal administration (e.g., intranasal or oral routes). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

If the pharmaceutical composition is administered orally, the pharmaceutical compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinized maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The pharmaceutical compositions may be administered by and formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions, or emulsions in oily or aqueous vehicles, and may contain one or more formulation agents such as suspending, stabilizing, or dispersing agents. Alternatively, the active ingredient may be in powder form for constitution with a suitable vehicle (e.g., sterile pyrogen-free water) before use. The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The pharmaceutical compositions may be formulated as neutral or salt forms. Pharmaceutically acceptable salts include those formed with anions such as those derived from hydrochloric, phosphoric, acetic, oxalic, tartaric acid, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachet indicating the quantity of active agent. Where the mode of administration is infusion, compositions can be dispensed with an infusion bottle containing sterile pharmaceutical grade water or saline. Where the mode of administration is by injection, an ampoule of sterile water for injection or saline can be provided so that the ingredients may be mixed prior to administration.

The pharmaceutical compositions may be in a variety of forms. These include, for example, liquid, semi-solid and solid dosage forms, such as liquid solutions (e.g., injectable or infusible solutions), dispersions or suspensions, tablets, pills, powders, liposomes, or suppositories. The preferred form depends on the intended mode of administration and therapeutic application.

In certain embodiments, modulator of the receptor, may be orally administered, for example, with an inert diluent or an assimilable edible carrier. The modulator of the receptor (and other ingredients, if desired) may also be enclosed in hard- or soft-shell gelatin capsules, compressed into tablets, or incorporated directly into the subject’s diet. For oral therapeutic administration, the modulator of the receptor may be incorporated with excipients and used in the form of ingestible tablets, buccal tablets, troches, capsules, elixirs, suspensions, syrups, wafers, and the like. To administer the modulator of the receptor by other than parenteral administration, it may be necessary to coat the modulator of the receptor with, or co-administer the modulator of the receptor with, a material to prevent its inactivation.

Modulators of the receptor can be used alone or in combination to treat the neurological and/or mental health condition. It should further be understood that the combinations are those combinations useful for their intended purpose.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### EXAMPLES

### Example 1

### Sensing Sugar in the Gut

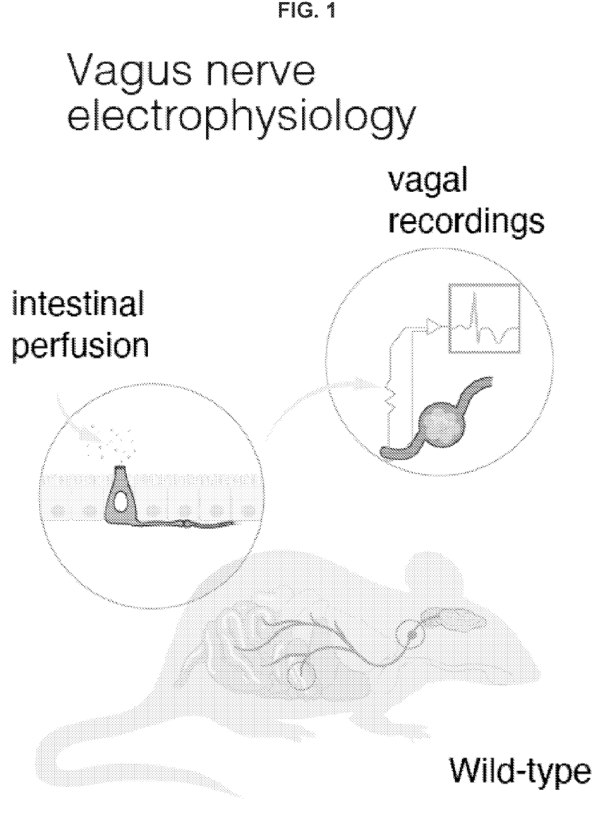

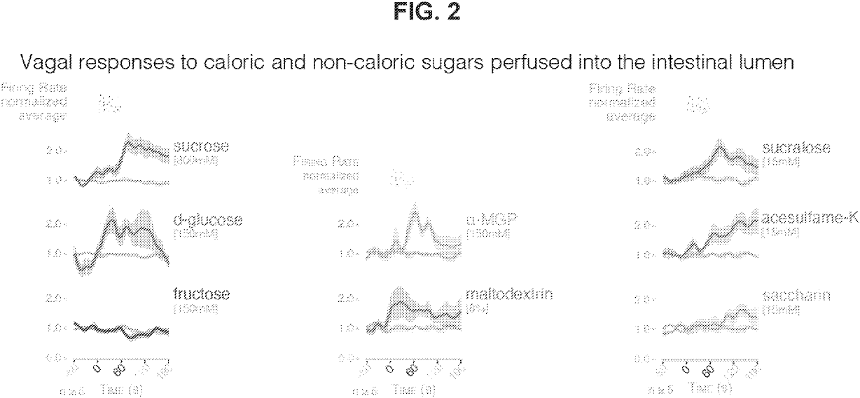

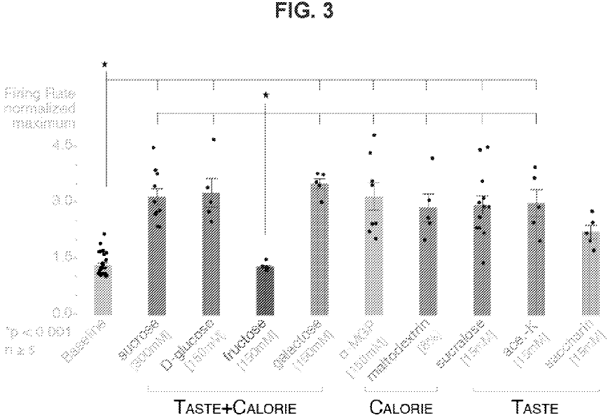

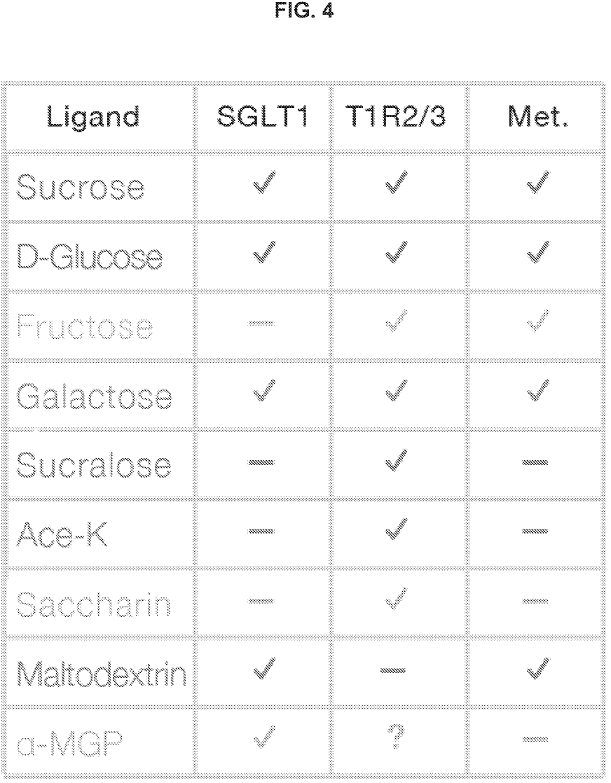

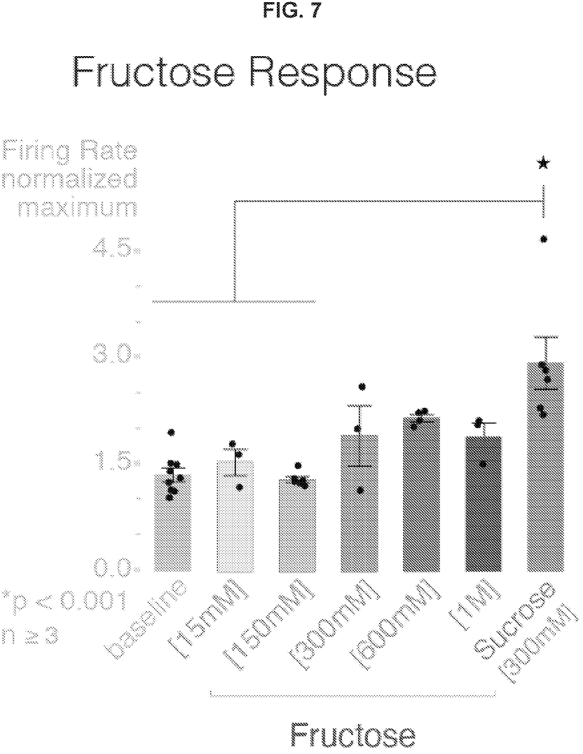

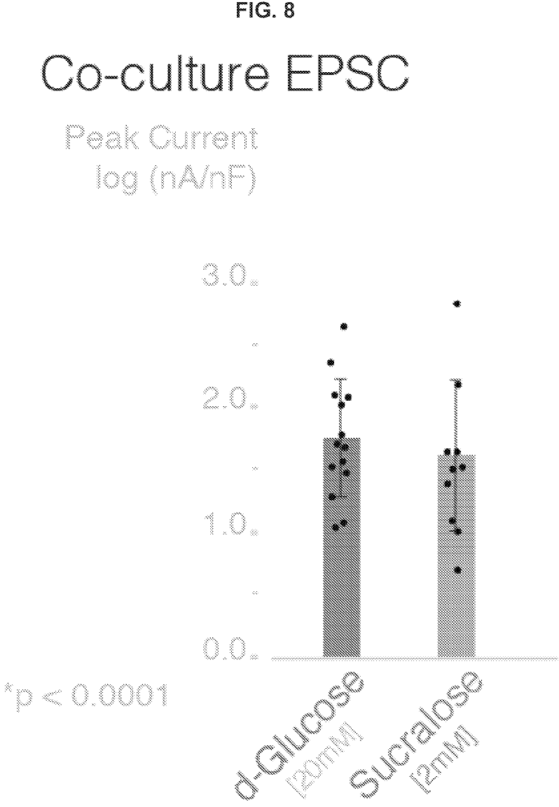

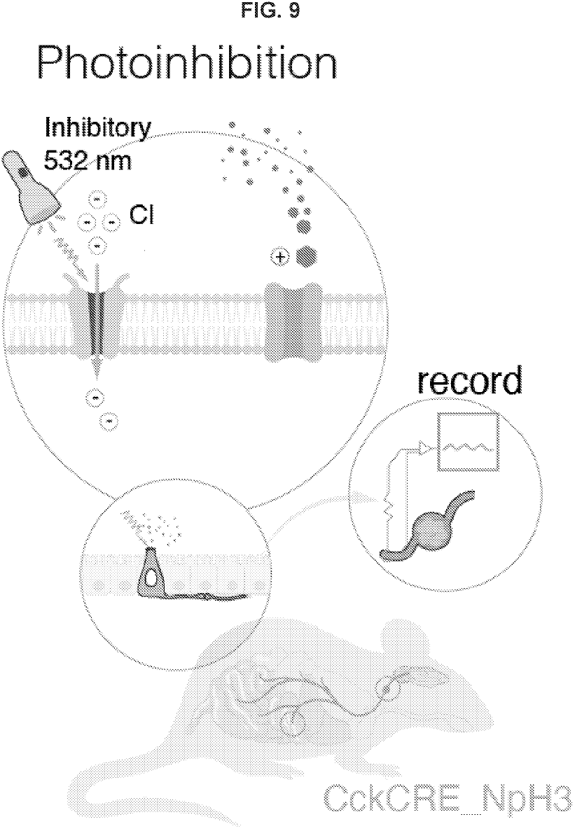

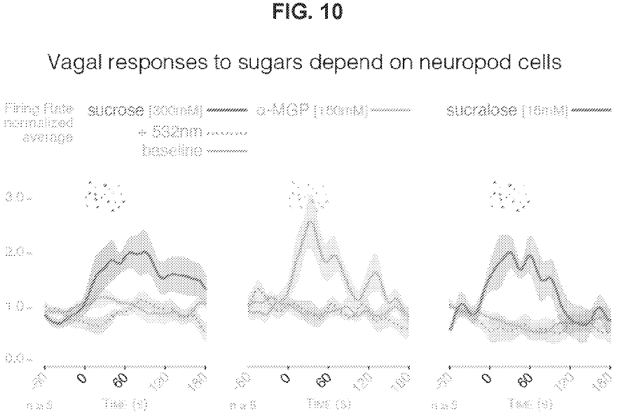

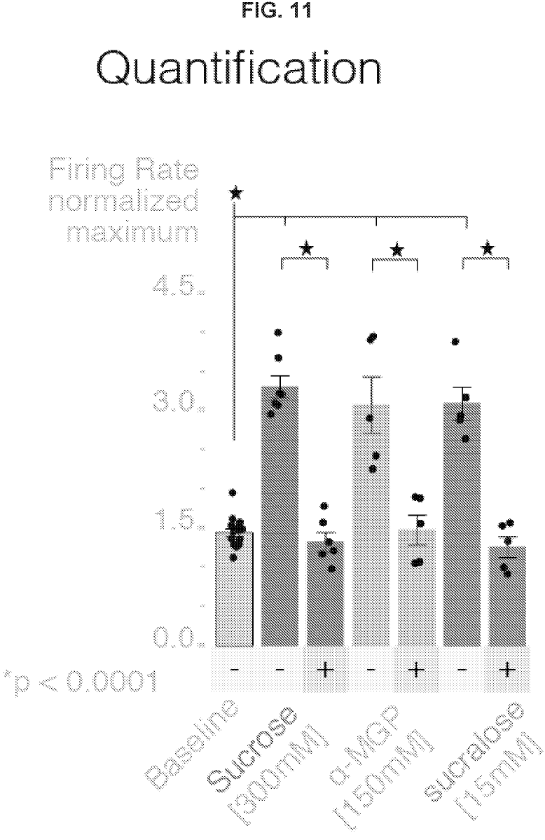

Using whole nerve electrophysiology of the cervical vagus nerve, it was confirmed that vagal activity increases in the presence of luminal sugars. Kaelberer et al., Science 361 (6408) (2018). The proximal duodenum was perfused with sugars, thus bypassing any gustatory or gastric activation. Sugars were tested based on the following properties: both caloric content and sweet taste (sucrose [300 mM], D-glucose [150 mM], fructose [150 mM], galactose [150 mM]), calorie-only (maltodextrin [8%], alpha-methylglucopyranoside (α-MGP) [150 mM]), or taste-only (sucralose [15 mM], acesulfame-K [15 mM], saccharin [15 mM]) (FIG. 1, FIG. 2, FIG. 3, FIG. 4). All of the sugars tested significantly (n ≥ 5, p < 0.001) increased vagal firing except saccharin and fructose (FIG. 3; osmolarity and peritoneal controls (Kaelberer et al., Science 361(6408) (2018)). However, fructose did appear to stimulate a blunted response at higher concentrations (600 mM and 1 M) (n = 3-5, n.s.) (FIG. 7 and FIG. 8). Next, the contribution of neuropod cells to the vagal response to caloric and non-caloric sugars was determined. A mouse in which small intestinal CCK+ cells express the inhibitory light activated channel eNphR3.0 (halorhodopsin) (Cck_Halo) was bred, then 532 nm intraluminal light was used to silence neuropod cells while perfusing sugar into the duodenum (FIG. 9). Intraluminal application of the 532 nm laser eliminated the vagal response to sucrose [300 mM], α-mgp [150 mM], and sucralose [15 mM] (n ≥ 5, p < 0.001) (FIG. 10 and FIG. 11; laser activation and wild-type controls). Thus, neuropod cells are required to sense both the calorie and taste properties of sugar and communicate these signals onto vagal neurons.

### Example 2

### Neuropod Cells can Distinguish Sugars

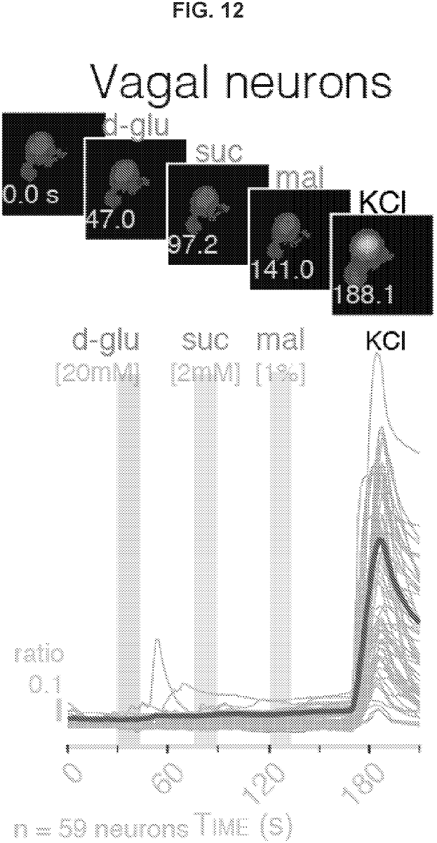

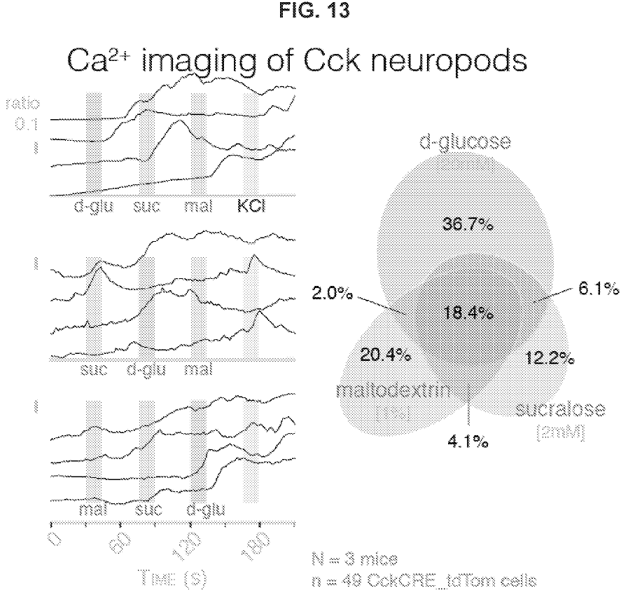

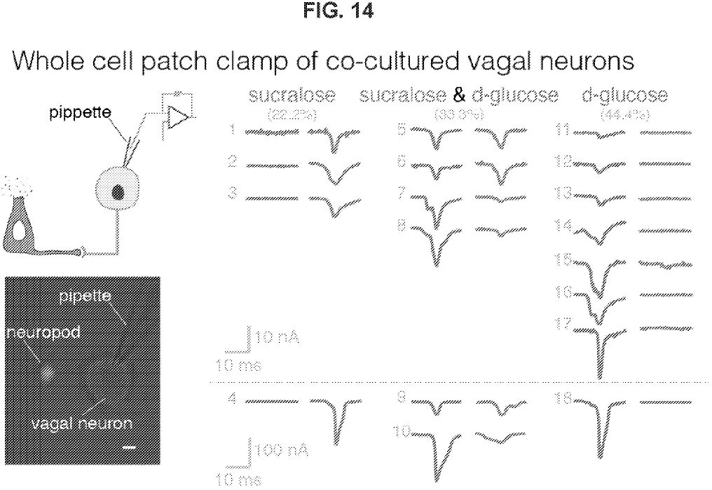

Dissociated vagal neurons were cultured to test whether the neurons are able to sense the different sugars. The neurons were stimulated with three consecutive sugars (D-glucose [20 mM], sucralose [2 mM], and maltodextrin [1%]) followed by KCI [50 mM] as an activity control. Only 2 of 59 neurons had a small response to d-glucose, while no neurons responded to the other sugars (FIG. 12). To determine how individual neuropod cells respond to taste and calorie properties, cultured Cck_tdTomato+ cells were stimulated with the same sugars, counterbalancing for order effect, and calcium activity was recorded using KCI [50 mM] as a final activity control. Of the 49 cells imaged, 18.4% responded to all types of sugar, 36.7% responded to only D-glucose, 20.4% to only maltodextrin, and 12.2% to only sucralose (n = 49 cells, n = 3 mice; FIG. 13). These data show that the neuropod cells are able to distinguish between sugars. Cck_tdTomato+ cells were co-cultured with nodose neurons to determine whether neuropod cells were transducing different sugar stimuli onto the same vagal neurons. Patch-clamp electrophysiology, in voltage-clamp, was used to record neuron activity in response to neuropod cell exposure to D-glucose [20 mM] and sucralose [2 mM] in alternating order (FIG. 14). From the 18 pairs recorded, neuropod cells transduced the sugar stimuli and elicited excitatory post-synaptic currents in connected neurons in response to both stimuli in 6 pairs, to only D-glucose in 8 pairs, and to sucralose alone in 4 pairs (FIG. 14). Thus, neuropod cells are heterogenous in their response and transduction of caloric and non-caloric stimuli to vagal neurons.

### Example 3

### Sensing Via Differential Nutrient Receptors

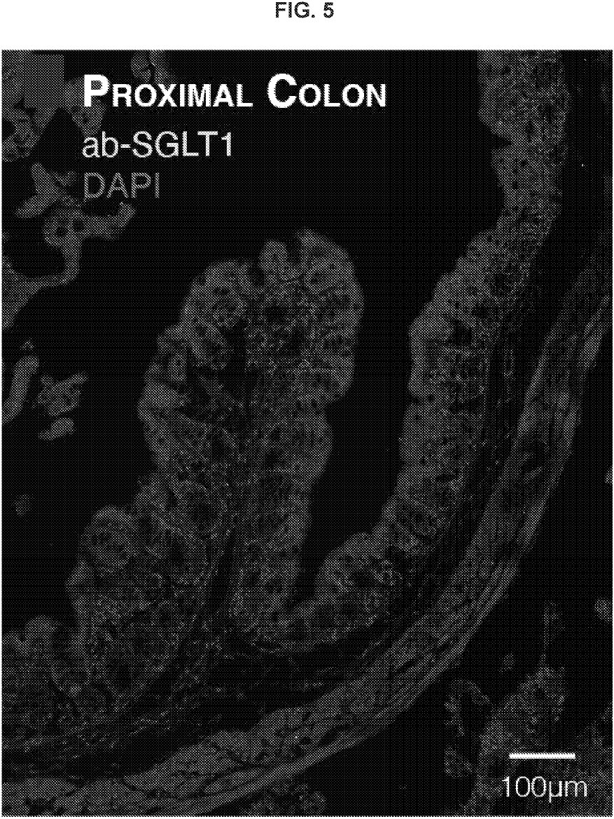

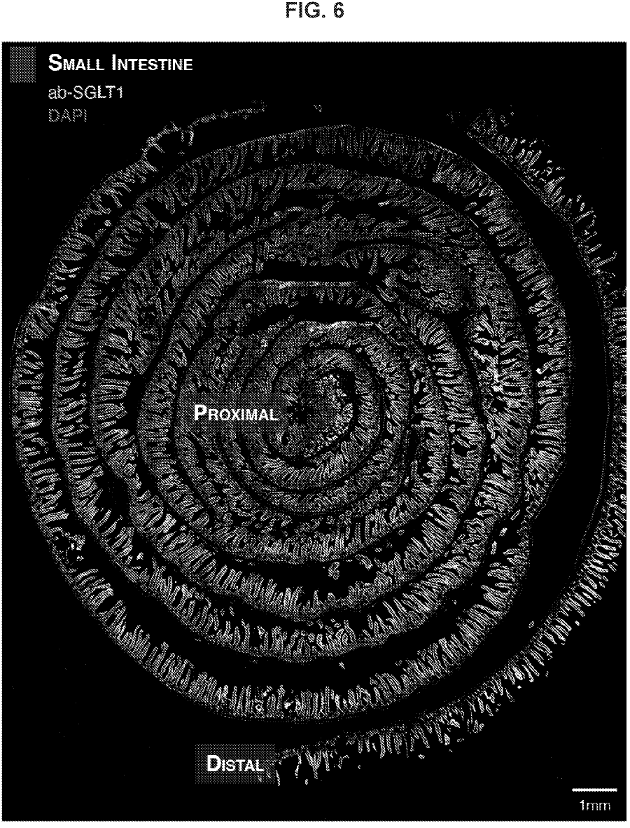

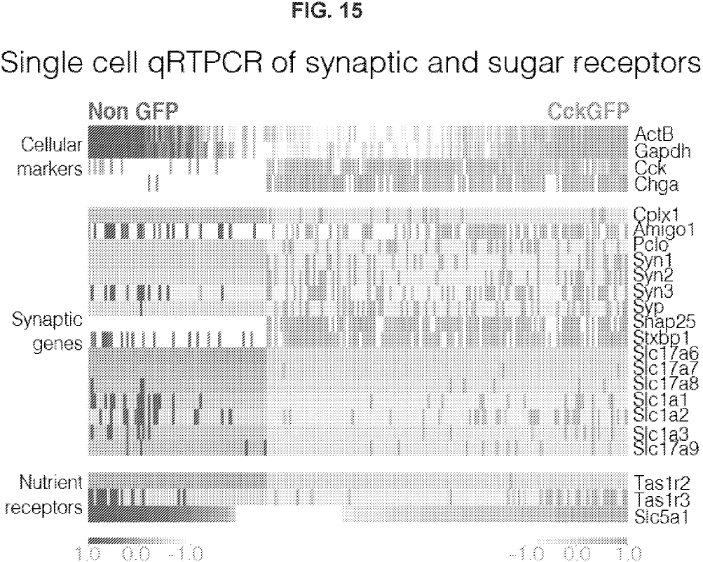

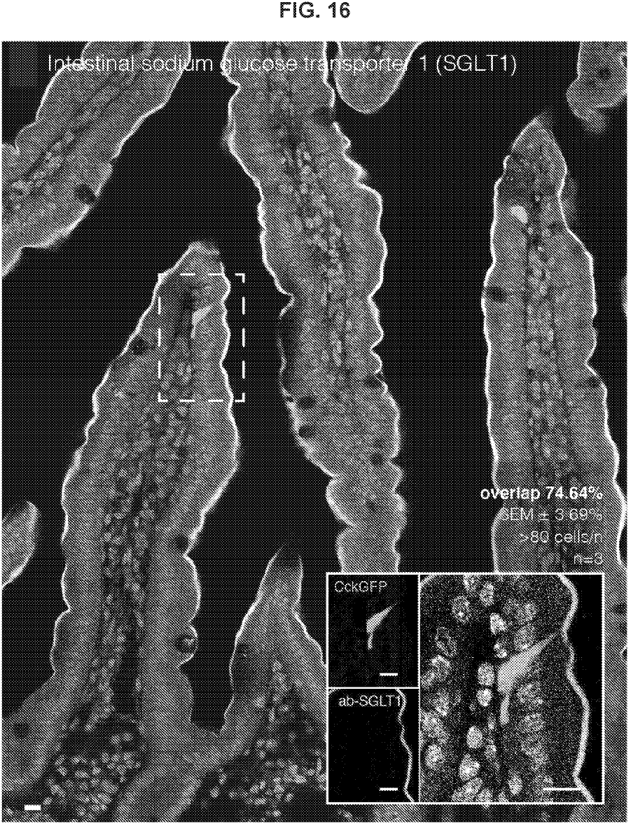

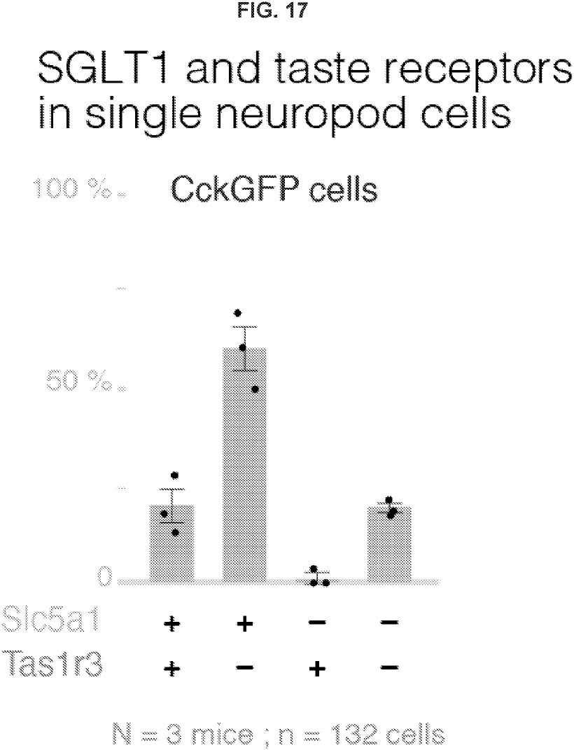

Enteroendocrine cells are known to express both sodium glucose like transporter (SGLT1) and sweet taste receptor sub-units T1R2 and T1R3. Daly, K. et al., Amer. J. Phisol.: Reg. Integr. Compar. Physiol. 303(2): R199-R208 (2012); Margolskee et al. USA Proc. Nat. Acad. Sci. 104(38): 15075-15080 (2007); Psichas et al., J. Clin. Invest. 125(3): 908-917 (2015). Whole sugars like D-glucose activate sweet taste receptors and enter the cell through SGLT1 where they undergo metabolism through glucokinase. Non-caloric sugars such as sucralose or saccharin activate sweet taste receptors, but do not enter through SGLT1. To isolate calorie sensing, maltodextrin, a natural compound commonly added to food which enters the cell through SGLT1 and undergoes metabolism, and α-mgp, a sugar analog that enters the cell through SGLT1 but is not further metabolized was used; neither are thought to activate sweet taste receptors (FIG. 4). To characterize the expression profile of nutrient receptors on neuropod cells specifically, single-cell quantitative PCR (qPCR) was performed on small intestinal epithelial cells of Cck-GFP+ mice. In CckGFP+ cells, transcripts for Slc5a1 (SGLT1) was detected in 78.8% of cells, Tas1r2 (T1R2) was detected in 0.76% cells, and Tas1r3 (T1R3) was detected in 46.2% cells (FIG. 15). The transcript expression data was confirmed for Slc5a1 (SGLT1) with immunohistochemistry. In the small intestine of CckGFP mice, 74.64% ± 3.69% CckGFP+ cells co-localize with SGLT1 protein in their brush border (n ≥ 80 cells per mouse, n = 3 mice; FIG. 16). The frequency of SGLT1 expressing Cck-GFP+ cells was consistent across the small intestinal length, and minimal SGLT1 expression was seen in the colon (FIG. 5 and FIG. 6). The low transcript expression by T1R2 is consistent with prior reports in pancreatic β-cells, suggesting that the T1R3 homodimer may be the primary receptor for sweet taste in gut sensor cells Kojima et al., Biol. Pharma. Bul. 38(5): 674-679 (2015); Zopun et al., J. Agri. Food Chem. 66(27): 7044-7053 (2018). Probing for overlap expression showed that 19.6% ± 4.34% cells expressed transcripts for both Slc5a1 and Tas1r3_{;} 60.1% ± 5.66% expressed only Slc5a1, few (1.24% ± 1.24%) expressed only Tas1r3_{:} and 19.1% ± 1.20% expressed neither (n = 3 mice, n = 132 CckGFP+ cells; FIG. 17).

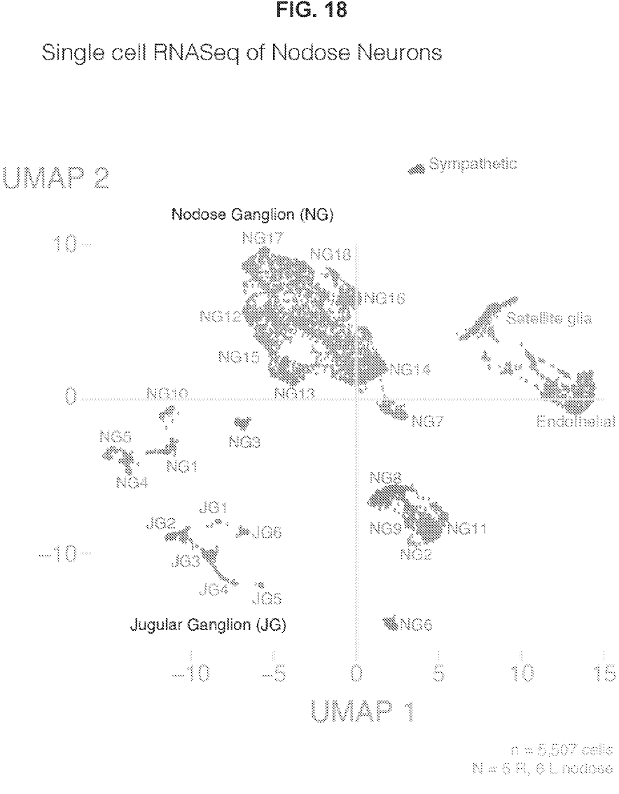

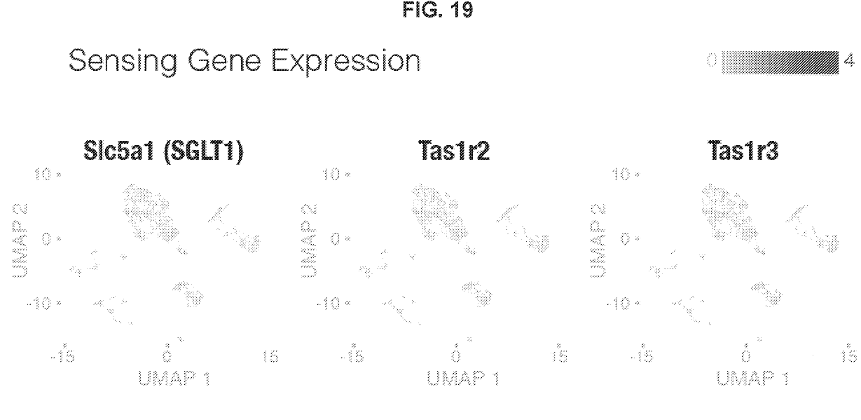

To confirm that CckGFP+ SGLT1 cells are neuropod cells, enrichment for synaptic transcripts was quantified. Slc5a1 + CckGFP+ cells were enriched in the synaptic adhesion genes Pvrll (fold-change 31.05) and Pvrl2 (fold-change 35.24) and the pre-synaptic genes Efnb2 (fold-change 81.65) and Cask (fold-change 30.19). To provide further evidence that these neuropod cells are directly sensing and transducing sugars, single cell RNA sequencing was performed on vagal nodose neurons and a likely gut-projecting cluster was isolated. In this and other clusters, nodose neurons did not express transcripts for Slc5a1, Tas1r2, or Tas1r3 (n = 5 right, 6 left nodose; FIG. 18 and FIG. 19).

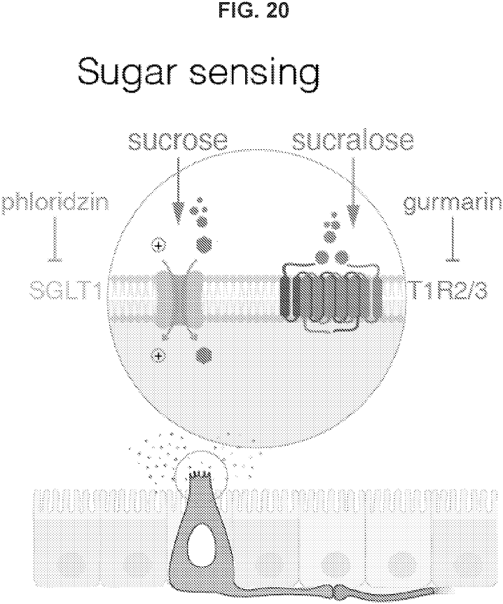

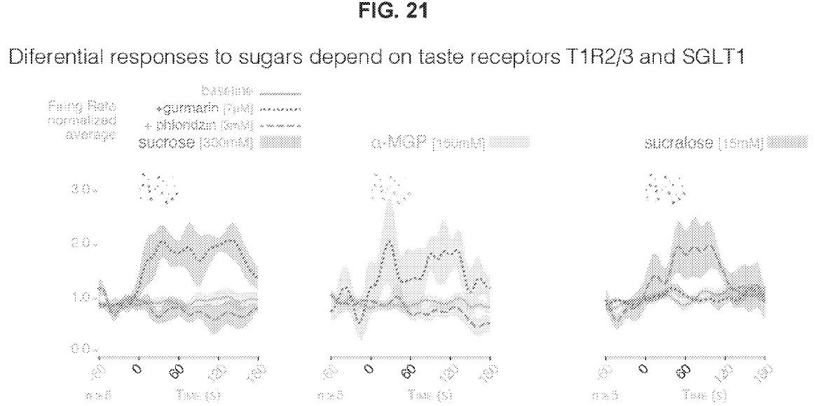

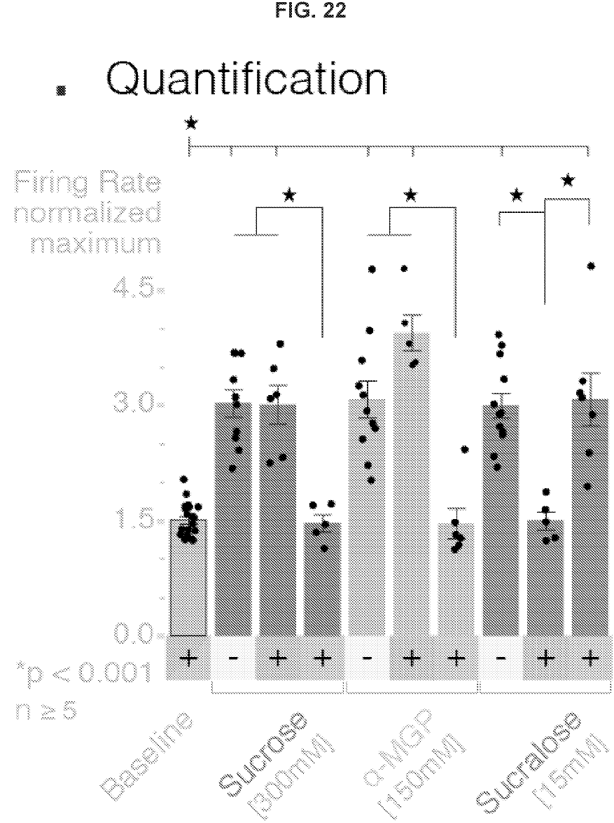

The contribution of these apical sensor proteins to the vagal response to caloric and non-caloric sugars was tested using receptor pharmacology in a whole nerve electrophysiology system (FIG. 20). Blocking the sweet taste receptor T1R2/3 with gurmarin [7 µM] abolished the increase in vagal firing rate elicited by sucralose [15 mM], but it did not affect the vagal response to sucrose at an iso-sweet concentration [300 mM] or to α-MGP [150 mM]. Conversely, vagal responses to both sucrose [300 mM] and α-MGP [150 mM] were abolished by inhibiting SGLT1 with phloridzin [3 mM]. The vagal response to sucralose [15 mM], however, was maintained in the presence of phloridzin [3 mM] (FIG. 21 and FIG. 22), showing neuropod cells use two distinct receptor mechanisms to transduce caloric and non-caloric sugar signals to the vagus nerve.

### Example 4

### Differential Neurotransmission of Sugars

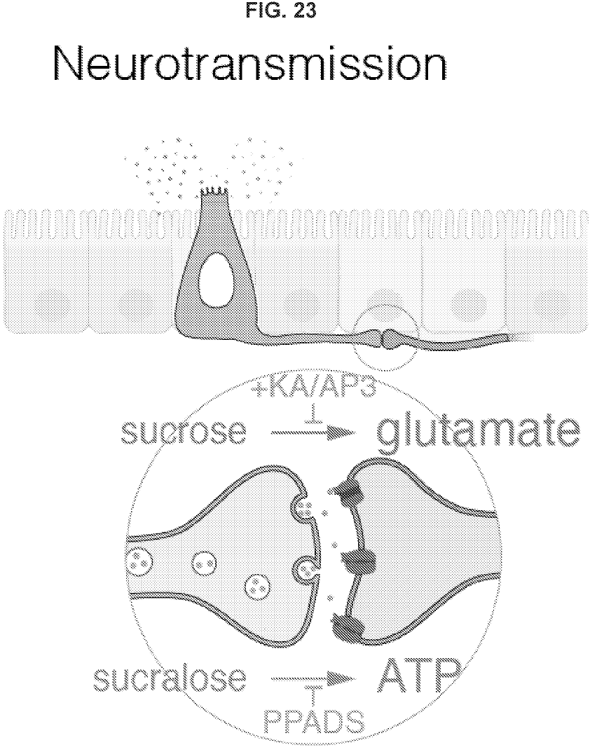

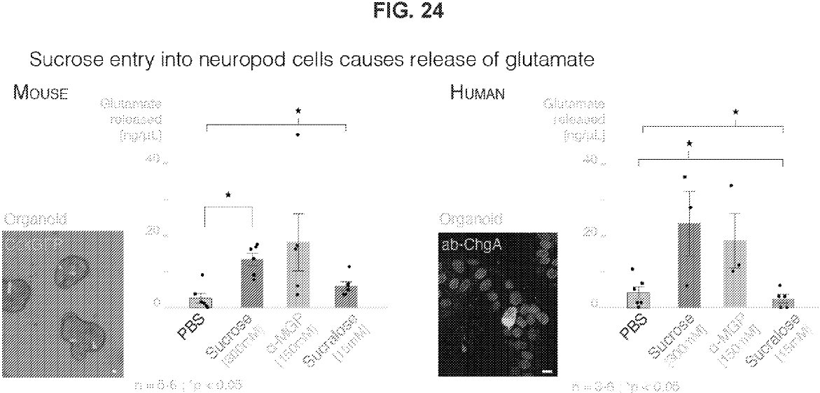

Slc5a1+ Cck-GFP cells express transcripts for the vesicular glutamate transporters Slc17a6, Slc17a7, Slc17a8 and the excitatory neurotransmitter transporters Slc1a1, Slc1a2, and Slc1a3 (FIG. 4). It was demonstrated that glutamate is the specific neurotransmitter for caloric sugar whereas ATP is a neurotransmitter for non-caloric sugar (FIG. 23). A glutamate release assay was performed on murine small intestinal organoids to determine if glutamate release was stimuli specific (FIG. 24-left). α-MGP was used for a caloric stimulus with no sweet taste, because it is not metabolized once it enters the cell and thus allowed for isolation of cell entry. After nutrient stimulation, the glutamate concentration in the supernatant was 2.632 ± 1.280 ng/µL for PBS control, 13.460 ± 1.724 ng/µL for sucrose [300 mM], 18.183 ± 7.904 ng/µL for α-MGP [150 mM], and 6.053 ± 1.169 ng/µL for sucralose [15 mM]. Glutamate release was significantly higher in response to sucrose and α-MGP, but not sucralose, compared to PBS (n = 5-6 trials in triplicate, n = 3 mice, p < 0.05; FIG. 24-left). Differential glutamate release was conserved in human tissue—using human duodenal organoids, sucrose [300 mM] and α-MGP [150 mM], but not sucralose [15 mM], elicited glutamate release, compared to PBS control (n = 3-6 trials, n = 1 human; p < 0.05; FIG. 24-right).

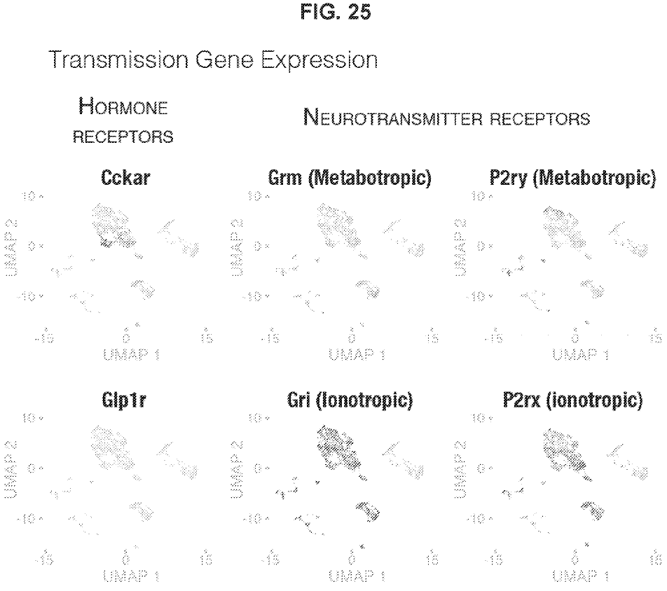

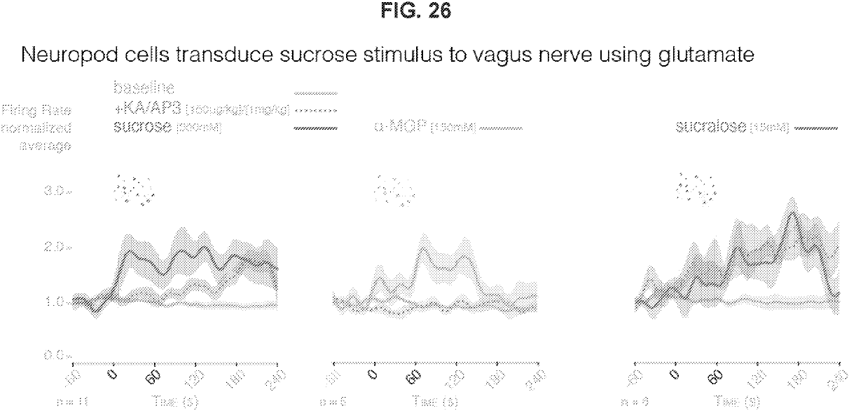

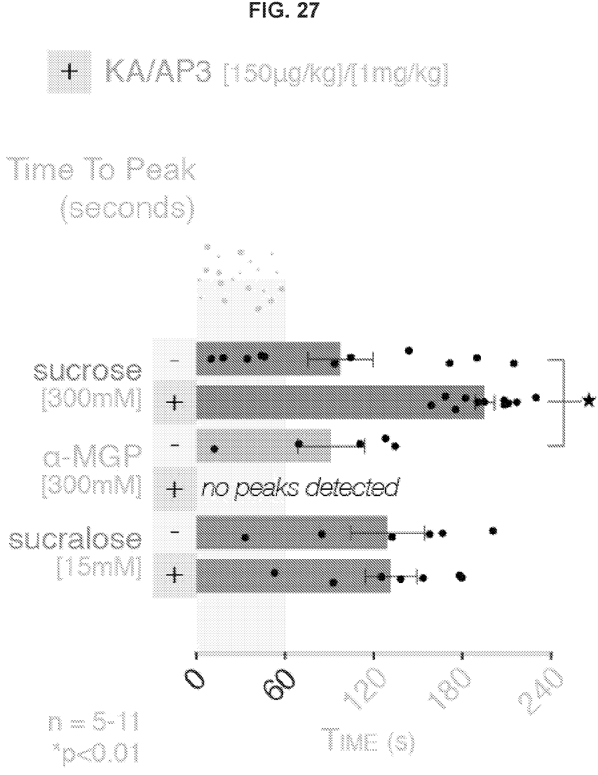

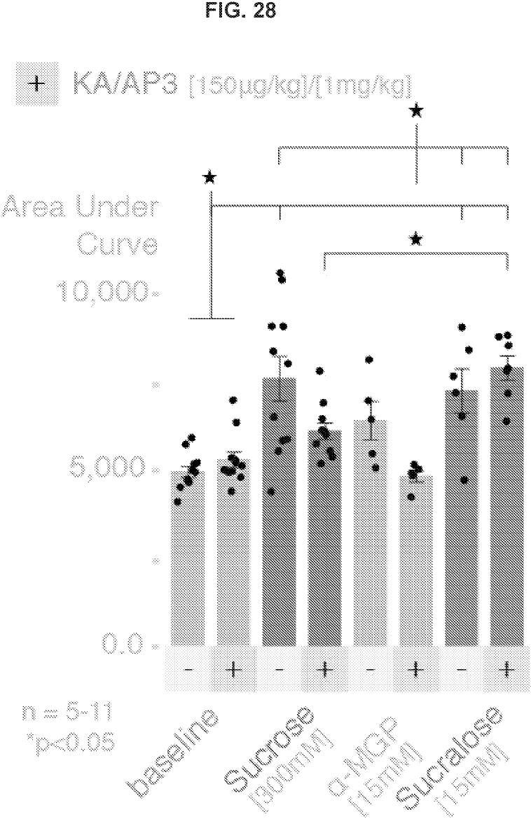

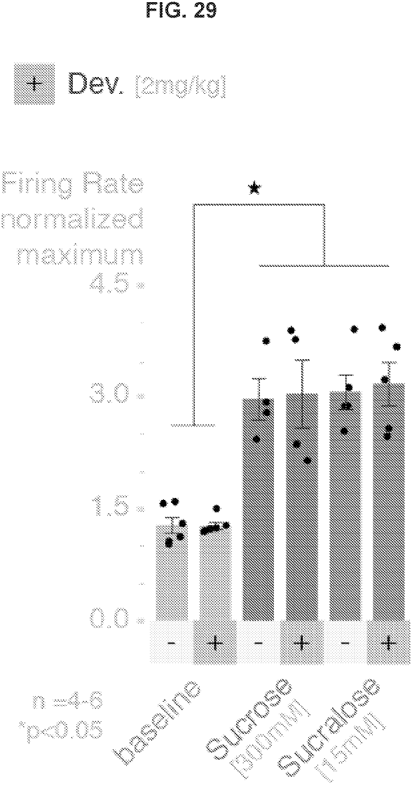

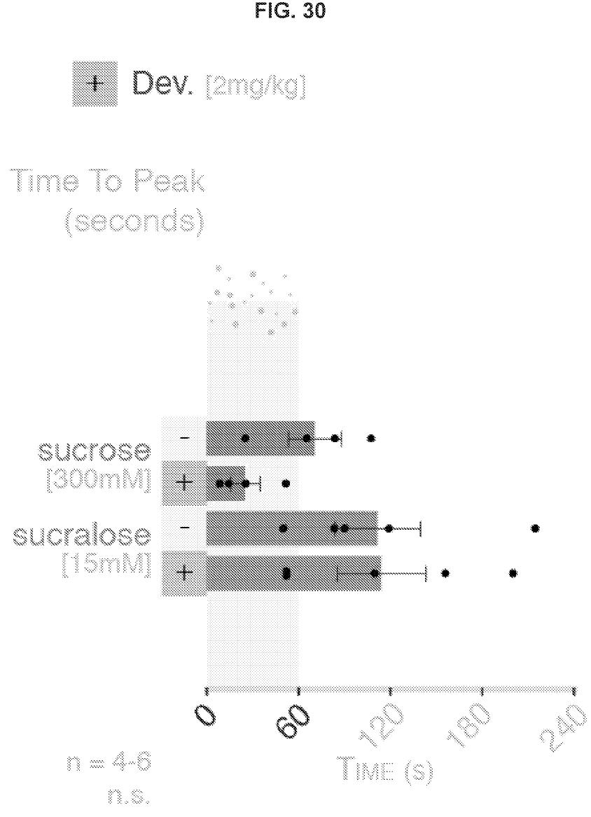

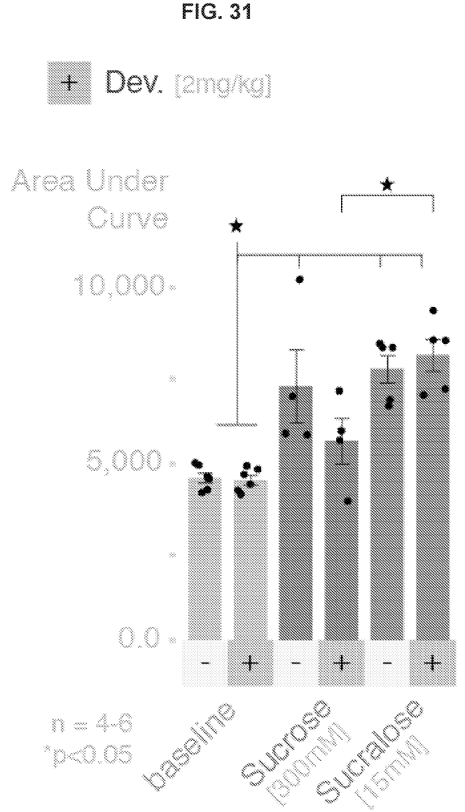

Single cell RNA sequencing of vagal nodose neurons showed that in addition to hormone receptors, glutamate and ATP receptors are also highly expressed (n = 5 right, 6 left nodose; FIG. 25). To identify the neurotransmitter for caloric and non-caloric sugars, vagal nerve recording was performed with specific receptor blockers. Glutamate receptors were inhibited with the ionotropic glutamate receptor inhibitor kynurenic acid [150 µg/kg] and the metabotropic glutamate receptor inhibitor AP3 [1 mg/kg] (KA/AP3). The rapid vagal response to sucrose [300 mM] was glutamate-dependent (FIG. 26, FIG. 27, and FIG. 28) while CCK-A receptor inhibition with devazepide [2 mg/kg] attenuated the prolonged response (FIG. 29, FIG. 30, and FIG. 31). Indeed, the vagal response to α-MGP [150 mM] was completely abolished by the glutamate receptor inhibitor cocktail (FIG. 26). Conversely, glutamate receptor inhibition did not affect the peak, timing, or magnitude of the vagal response to sucralose [15 mM], nor did Cck-A receptor inhibition with devazepide [2 mg/kg] (FIG. 26, FIG. 27, FIG. 28, FIG. 32, FIG. 26, FIG. 27, and FIG. 28). Thus, entry of caloric sugar (through SGLT1) is necessary and sufficient to drive glutamatergic transmission between neuropod cells and vagal neurons.

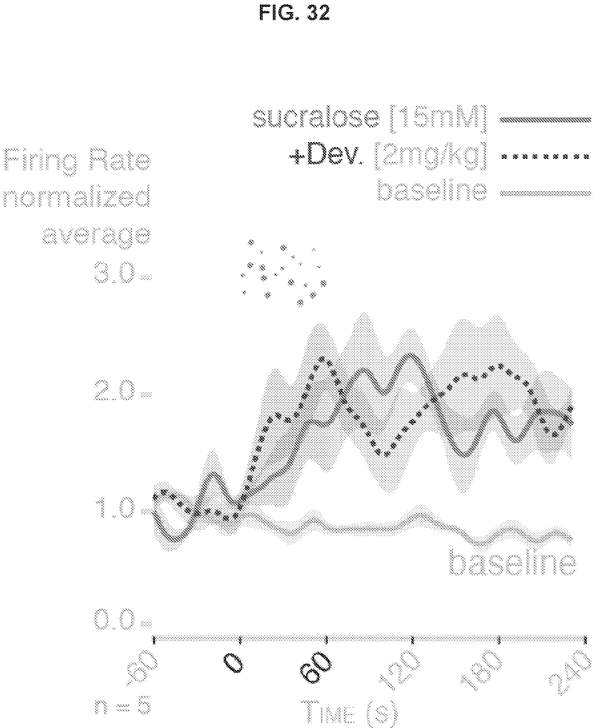

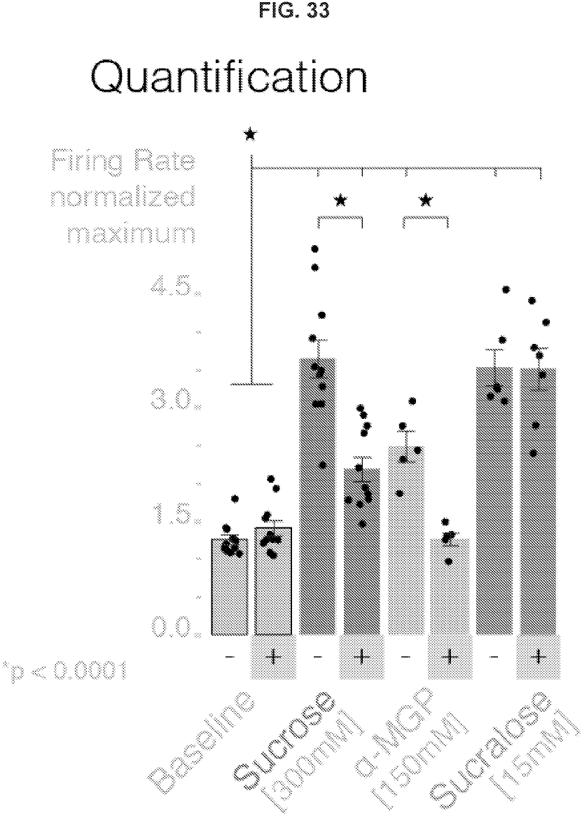

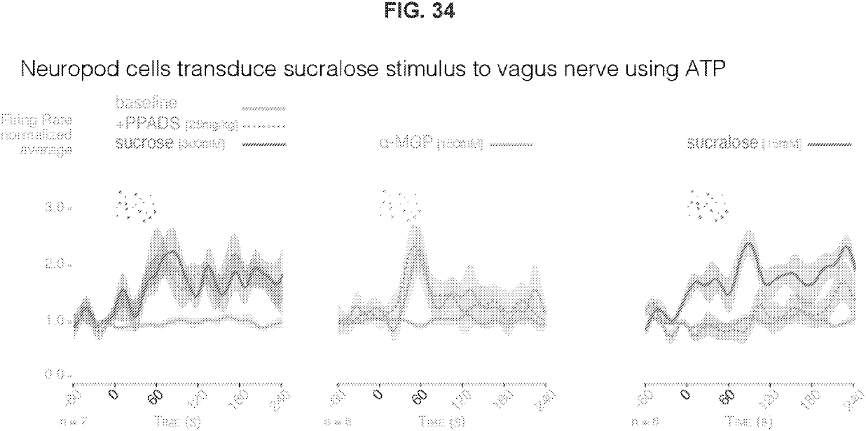

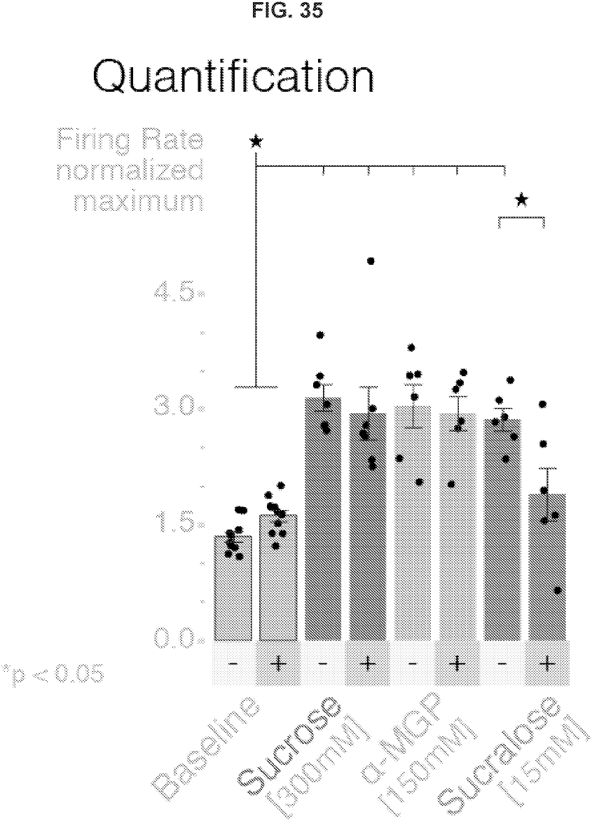

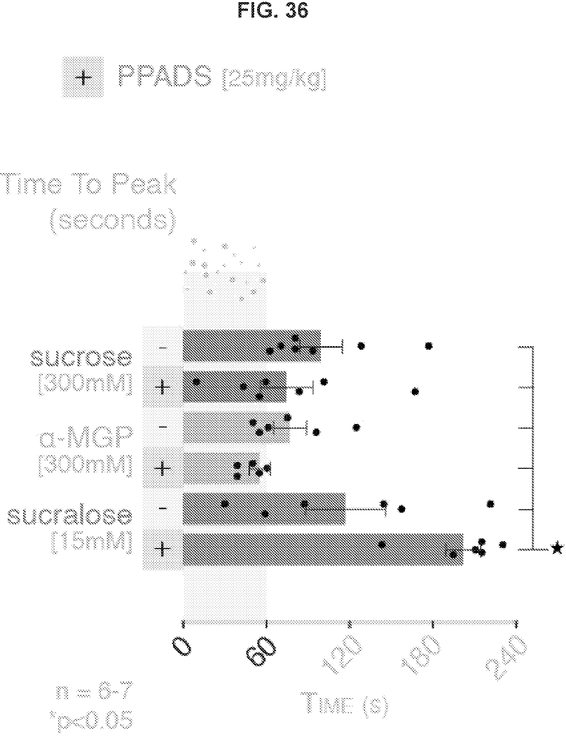

In gustatory sensation of sweet taste, ATP has been identified as a key neurotransmitter and, in ileal enteroendocrine cells, it has been shown to be co-released with the hormone GLP-1. Margolskee, J. Biol. Chem. 277(1): 1-4 (2002); Lu et al., Nature Com. 10(1): (2019). Single cell qPCR data showed a population of CckGFP+ cells that express the Slc17a9 transcript for the vesicular nucleotide transporter VNUT (FIG. 15). inhibiting P2 purinoreceptors with the non-selective antagonist PPADS demonstrated that ATP significantly attenuates the peak, speed, and magnitude of the vagal response to sucralose [15 mM], but not sucrose [300 mM] or α-MGP [150 mM] (FIG. 33, FIG. 36, and FIG. 37). These data show that neuropod cells are using different neurotransmitters to communicate the caloric content of sugars.

### Example 5

### A Choice for Sugar

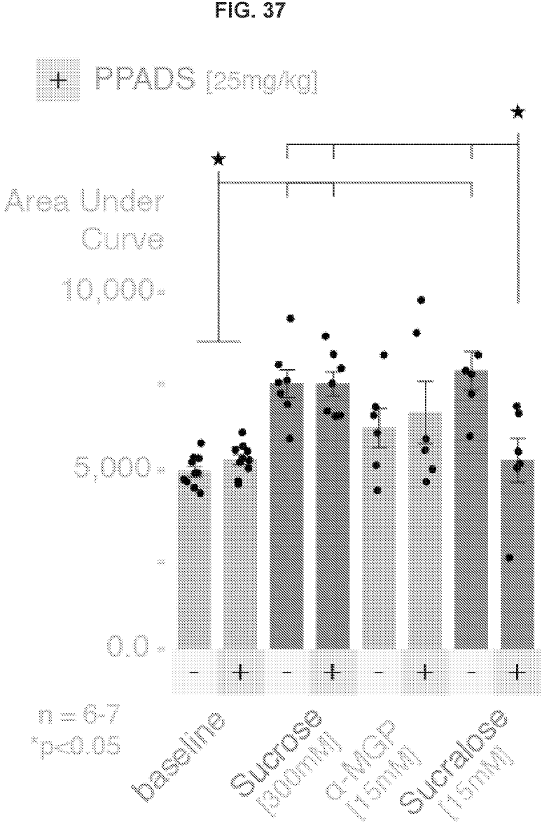

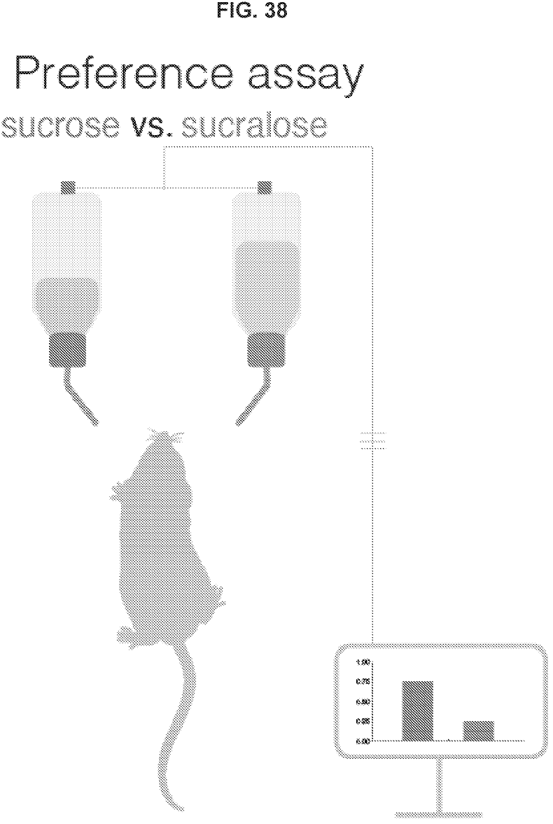

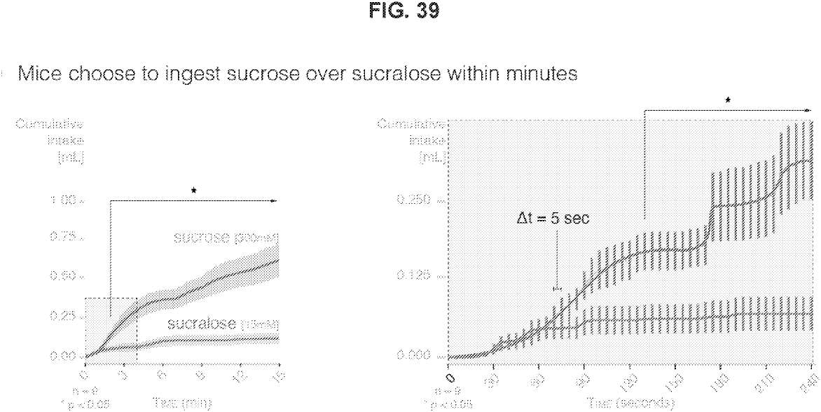

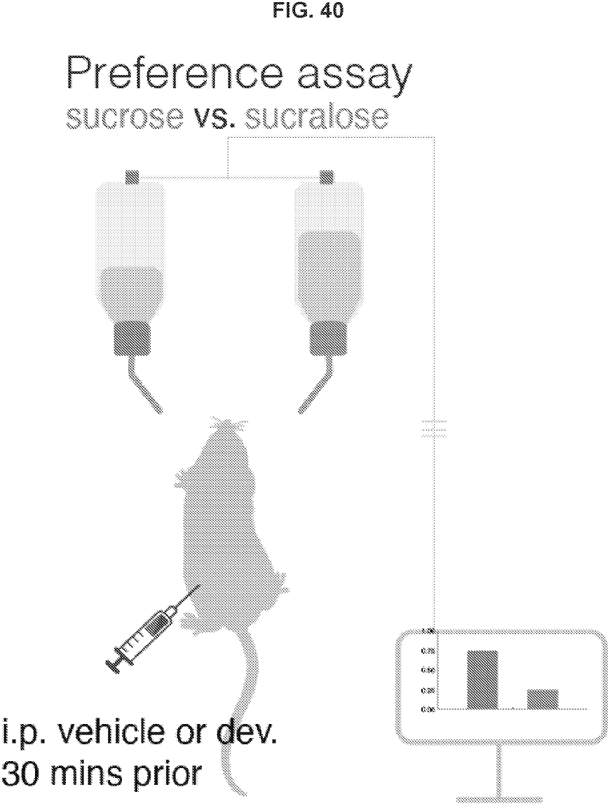

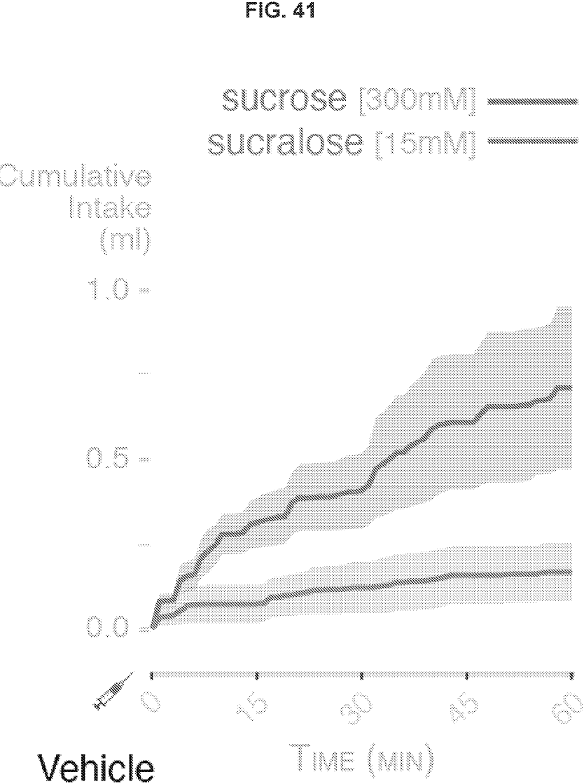

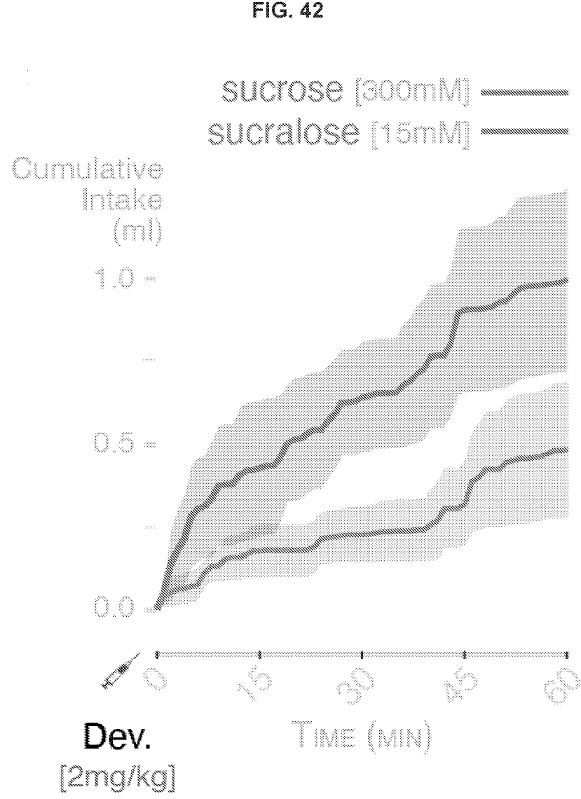

Given the choice between iso-sweet sucrose and sucralose, mice develop a robust preference for the caloric sugar. Domingos et al., Nature Neurosci. 14(12): 1562-1568 (2011); Sclafani and Ackroff, Physiol. Behav. 173: 188-199 (2017). Using an automated system, intake was measured from two bottles simultaneously at 5 second intervals and it was revealed that the preference for sucrose over sucralose does not occur instantaneously (FIG. 38 and FIG. 39). Rather, it took 2.083 minutes for experienced mice fasted for 1 hour to develop a significant preference for isosweet sucrose [300 mM] over sucralose [15 mM] (n = 9 mice, p < 0.05; FIG. 39). This time course corresponds to initial gastric emptying of liquids in fasted mice, thus suggesting a duodenal driver of sugar preference. Bennink et al., J. Nucl. Med. 44(7): 1099-1104 (2003); Poulakos and Kent, Gastroenterol. 64(5): 962-967 (1973). While involvement of the small intestinal gut hormone CCK has been suggested, prior studies have shown that inhibition of CCK-A receptors does not affect sugar preference. Perez et al., Pharmacol. Biochem. Behav. 59(2): 451-457 (1998). Here, it was confirmed that inhibition of CCK-A receptors with devazepide [2 mg/kg] injected intraperitoneally does not change preference for sugar over artificial sweetener (FIG. 40, FIG. 41, FIG. 42, and FIG. 43). Thus, a goal of this study was to determine if synaptic gut-brain neurotransmission drives sugar preference.

### Example 6

### Flexible Fiberoptic Development

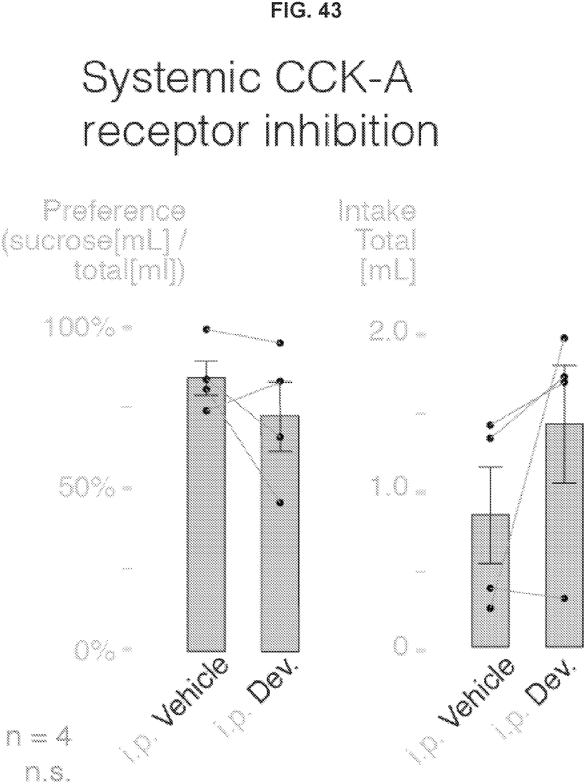

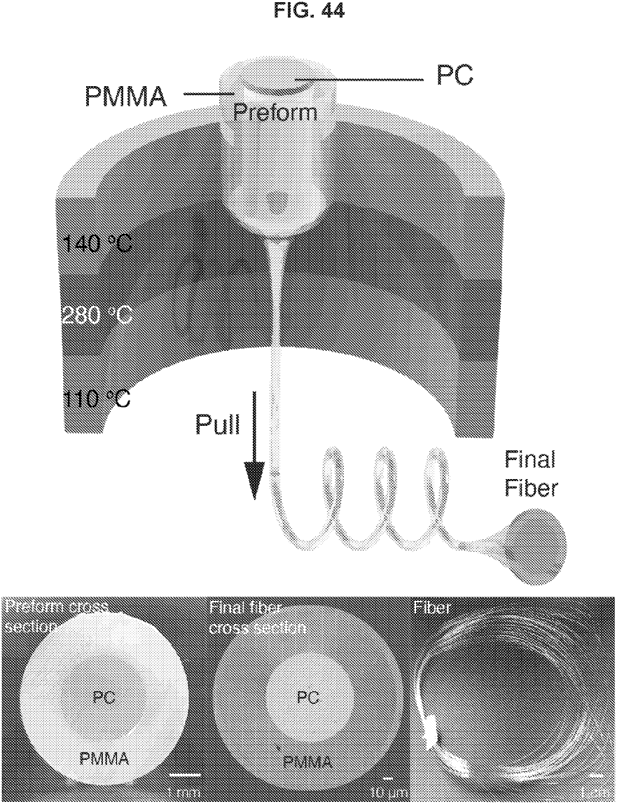

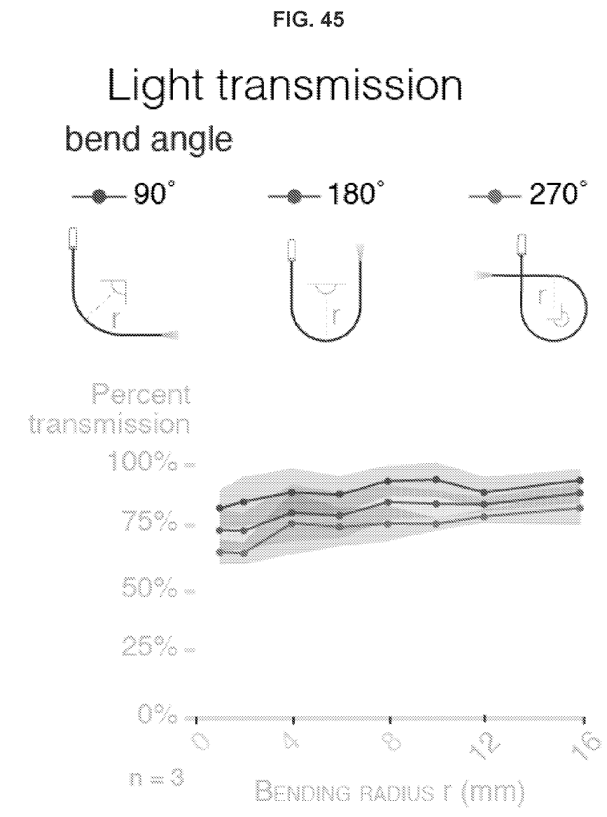

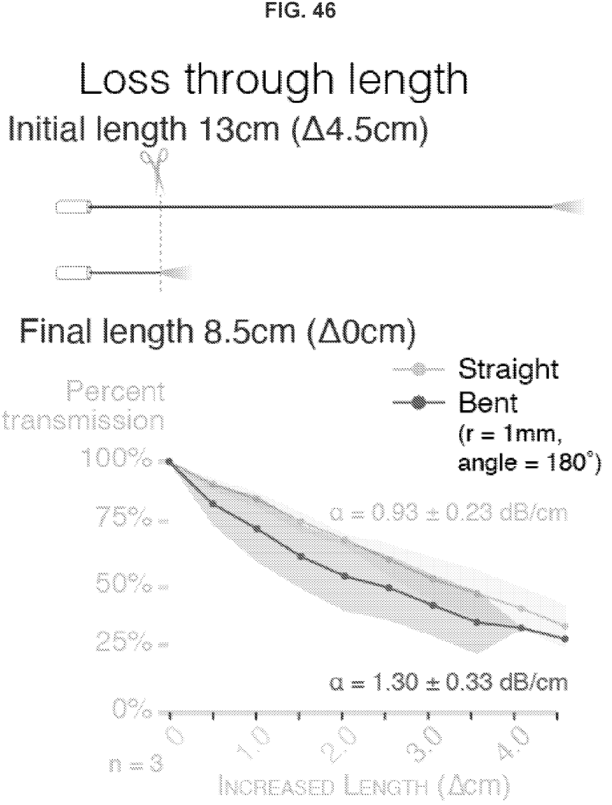

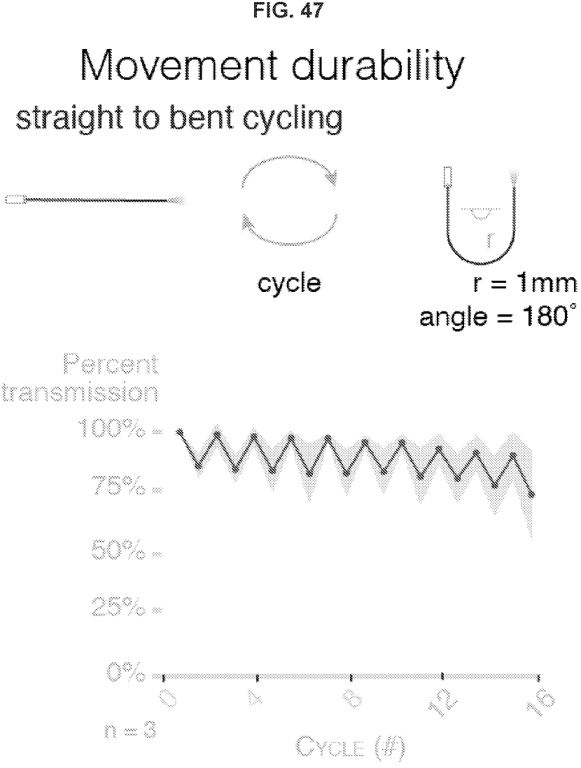

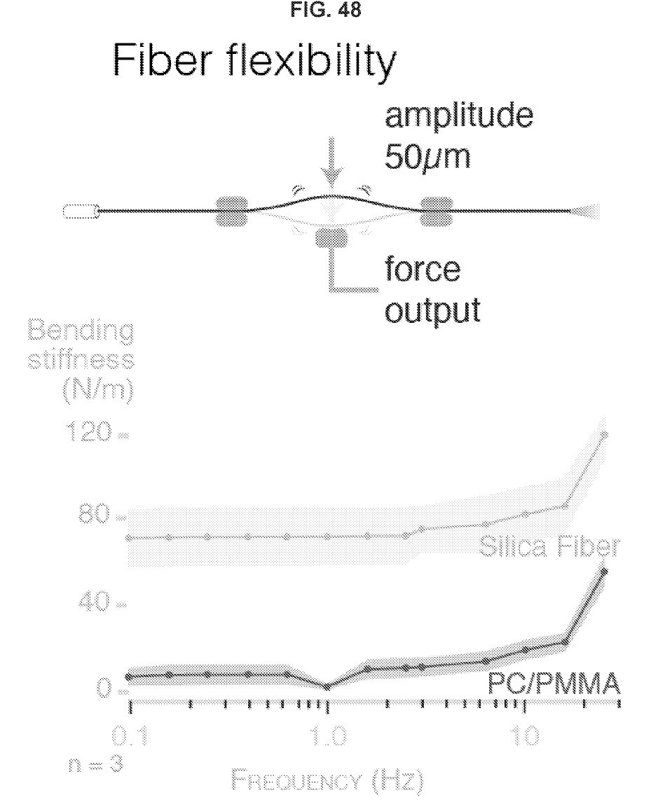

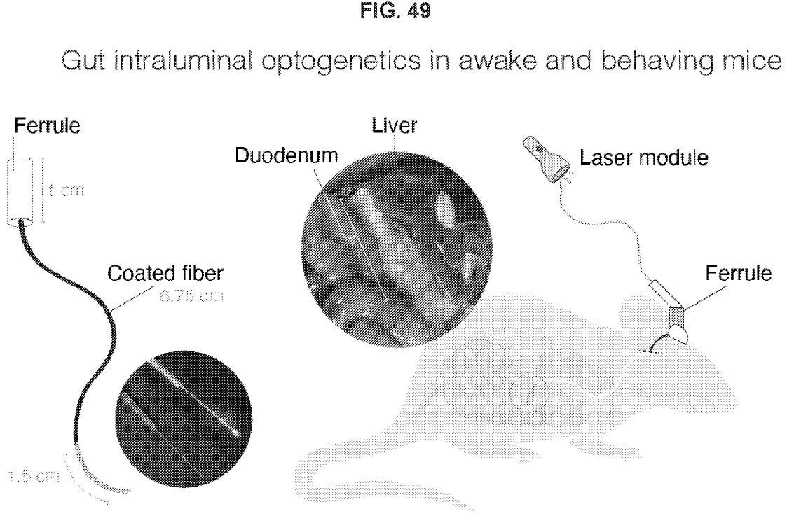

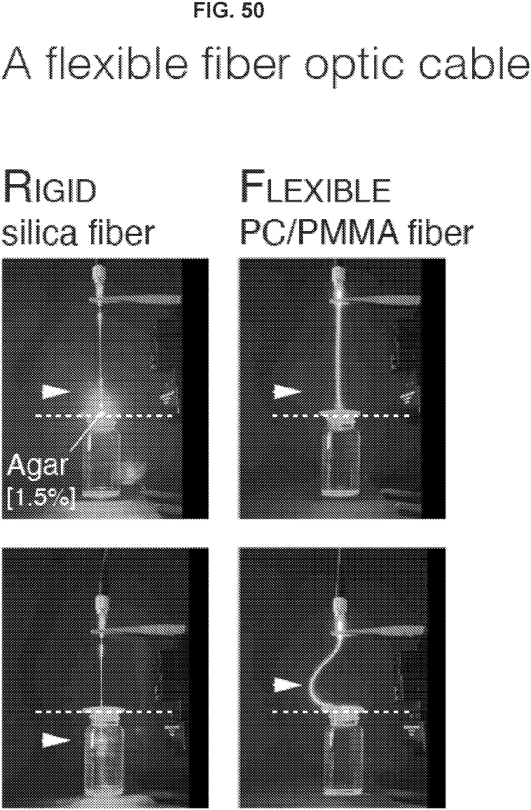

Although optogenetic tools have been widely used to probe brain circuits driving behavior, technical hurdles have limited their use in the periphery. The classic stiff fiberoptic cables implanted in the brain are at odds with the delicate intestinal lumen where flexibility is requisite. To determine if neuropod cells are the gut signal driving preference for caloric over non-caloric sugars, a novel flexible fiberoptic was developed to surmount the challenges of applying optogenetics to the gut. This PC/PMMA (core/cladding) fiber has three key properties: (1) small diameter (230 µm diameter) for minimal footprint within the intestinal lumen (FIG. 44), (2) light transmission at bending angles of up to 270° and minimal lossiness at extended lengths to allow for tunneling (FIG. 45 and FIG. 46), and (3) durability and flexibility at physiologic frequencies for preserved function weeks to months after implantation (FIG. 47 and FIG. 48). The fiber was enclosed in an opacified MicroRenathane® (Braintree Scientific Inc.) sheath to improve durability and avoid non-specific light application (FIG. 49). It is called the il"lumen"ator. While standard silica fibers penetrated a thin layer of agarose (1.5%), the flexible fiberoptic did not (FIG. 50). Over four weeks after implantation, the device’s functionality was assessed using vagal nerve recordings with the device in situ. As expected, 532 nm light emitted from the device in the duodenum inhibited the vagal response to 300 mM sucrose while 473 nm light had no effect on the response (FIG. 51 and FIG. 52).

### Example 7

### A Gut Sensor for Calories

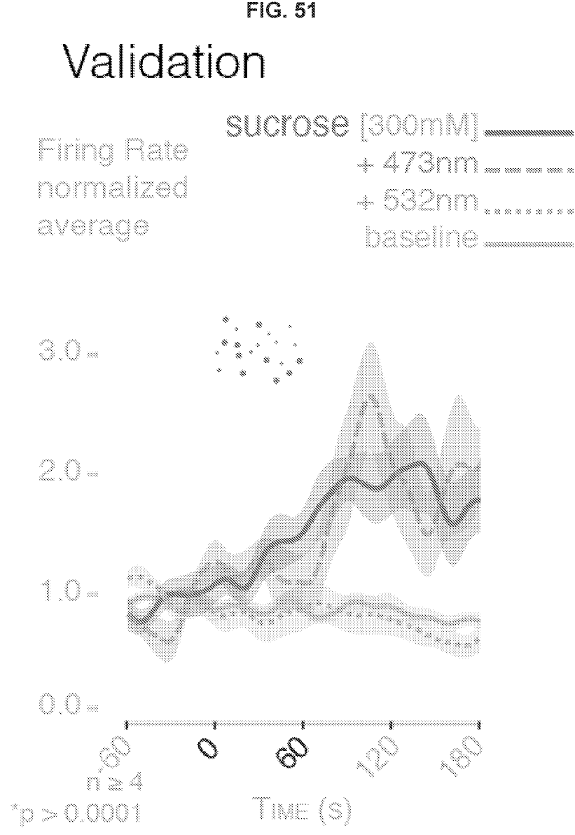

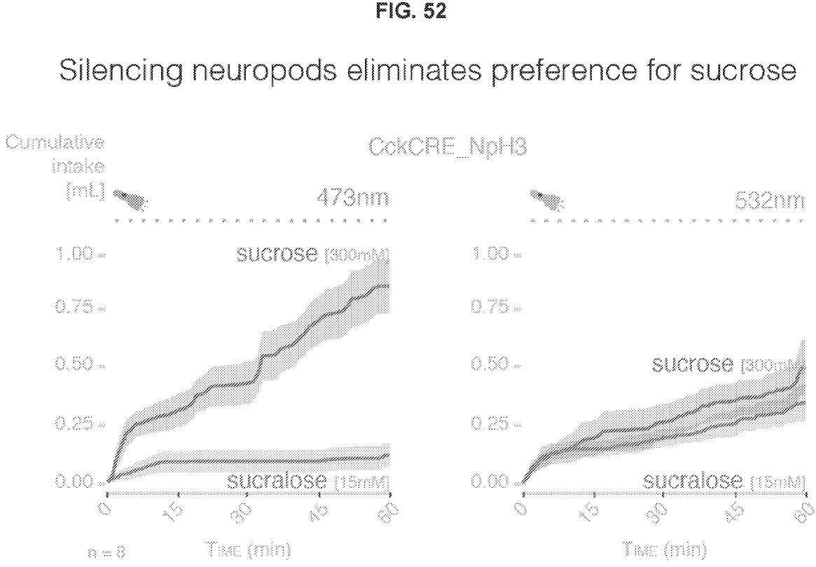

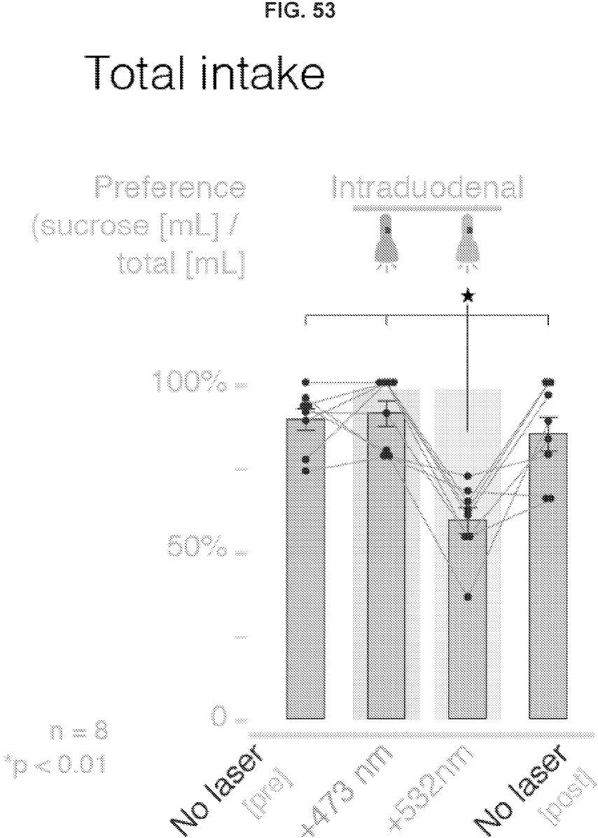

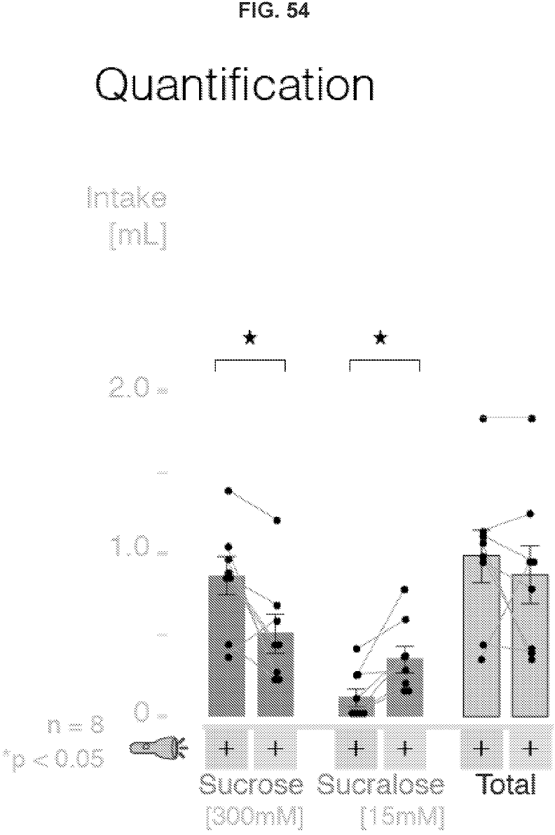

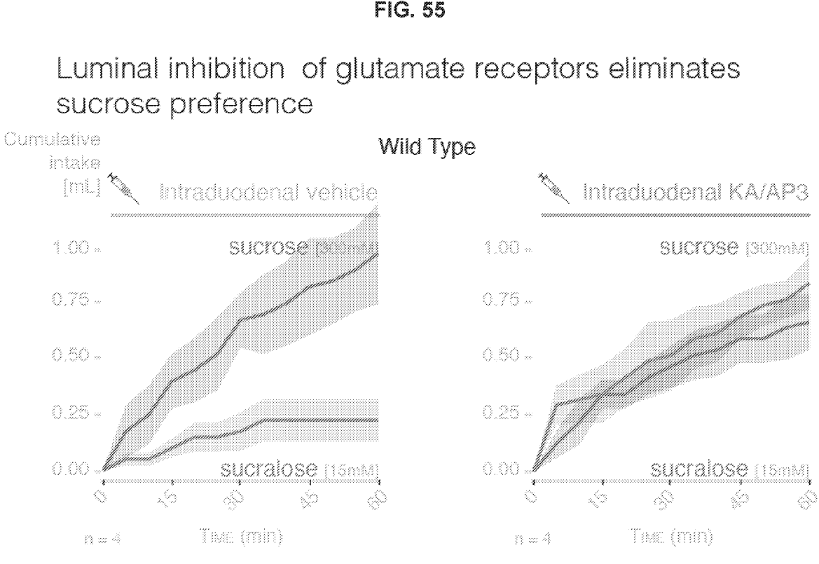

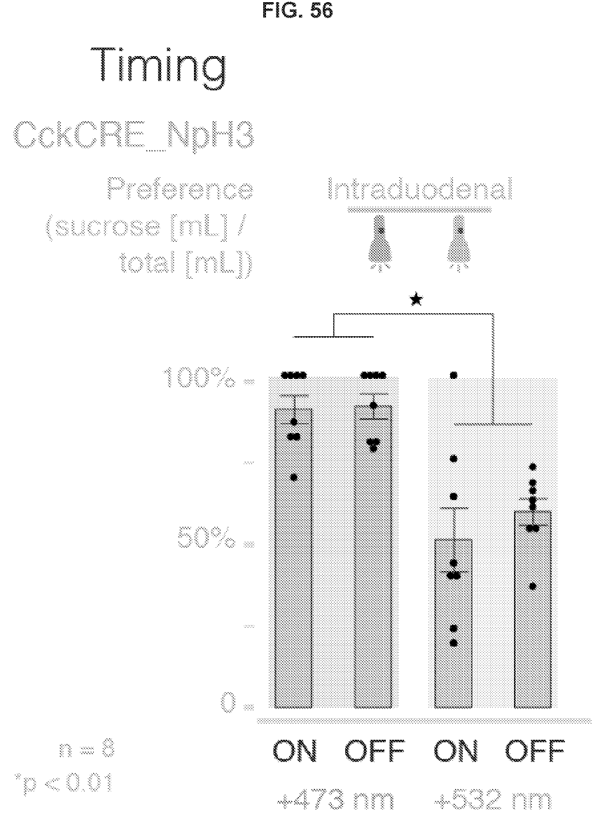

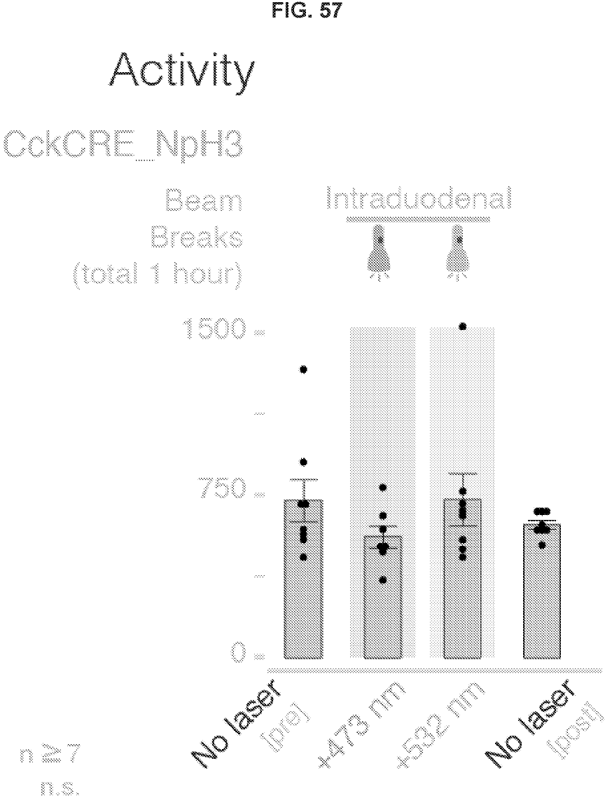

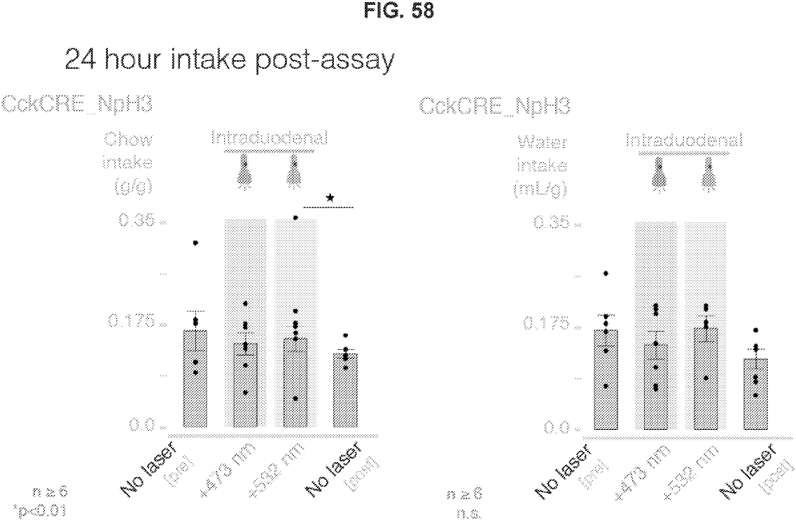

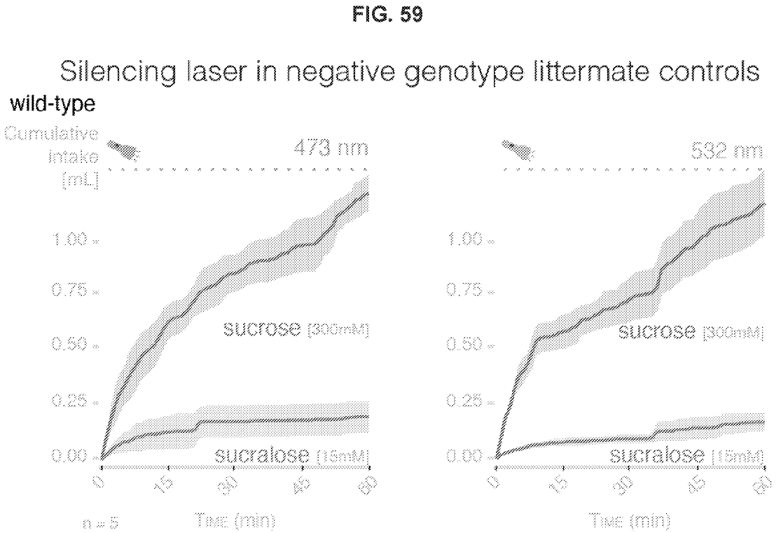

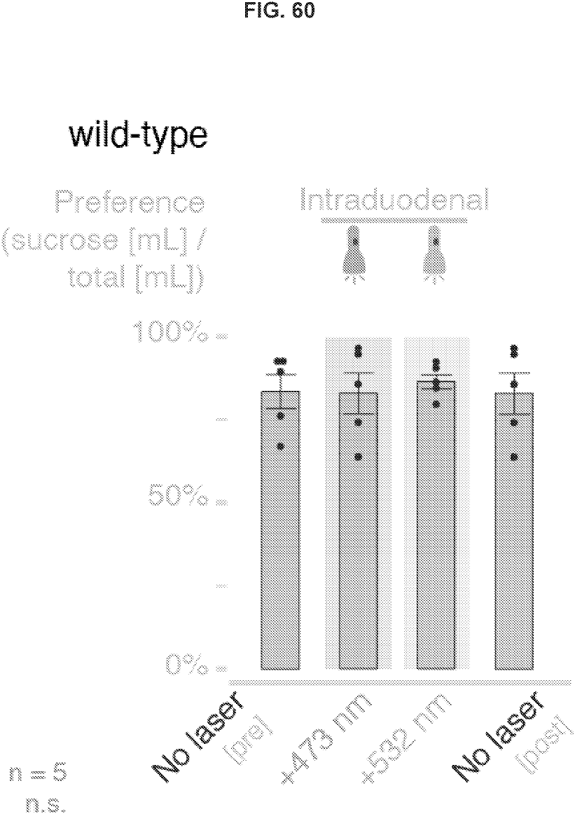

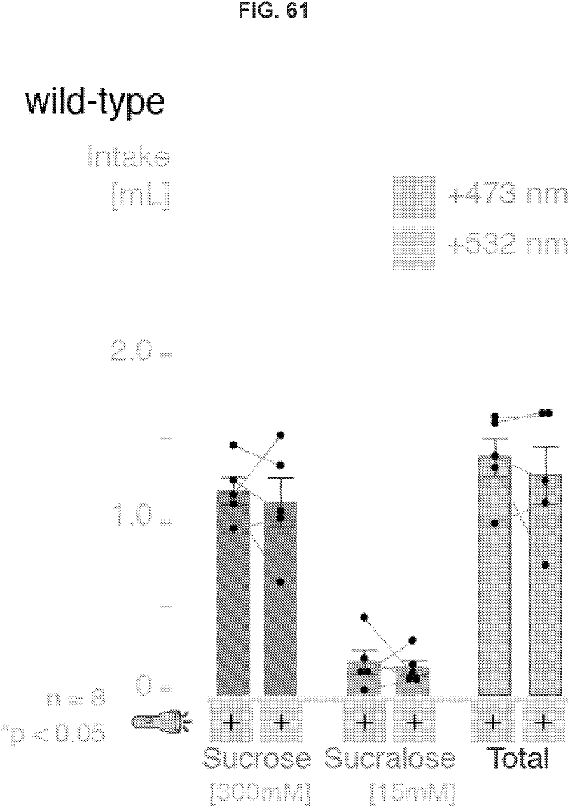

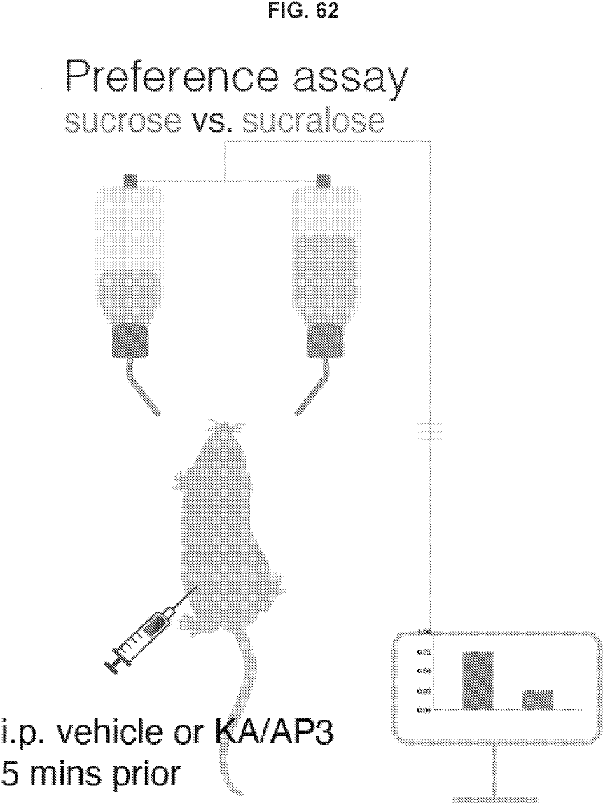

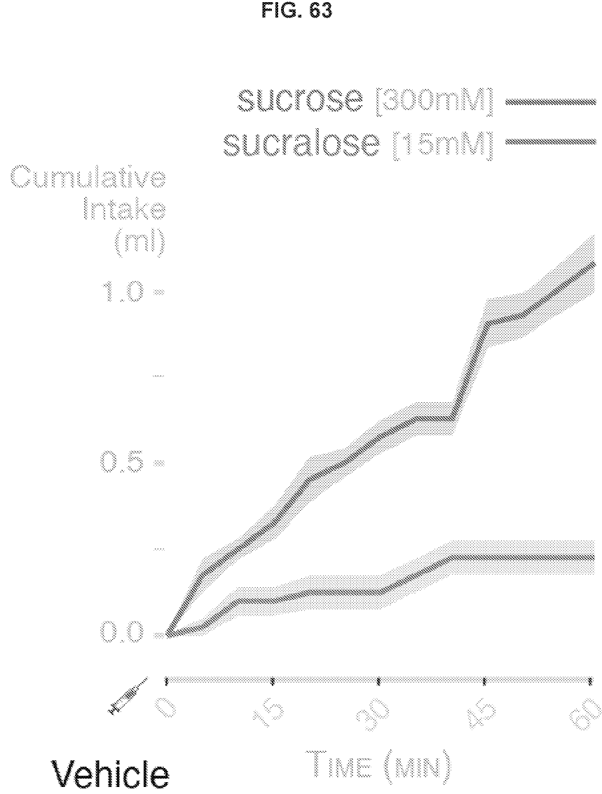

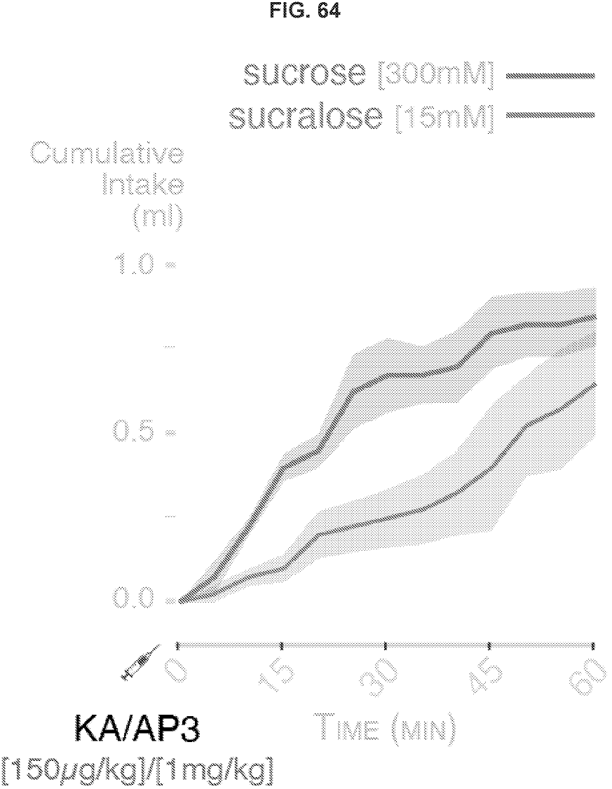

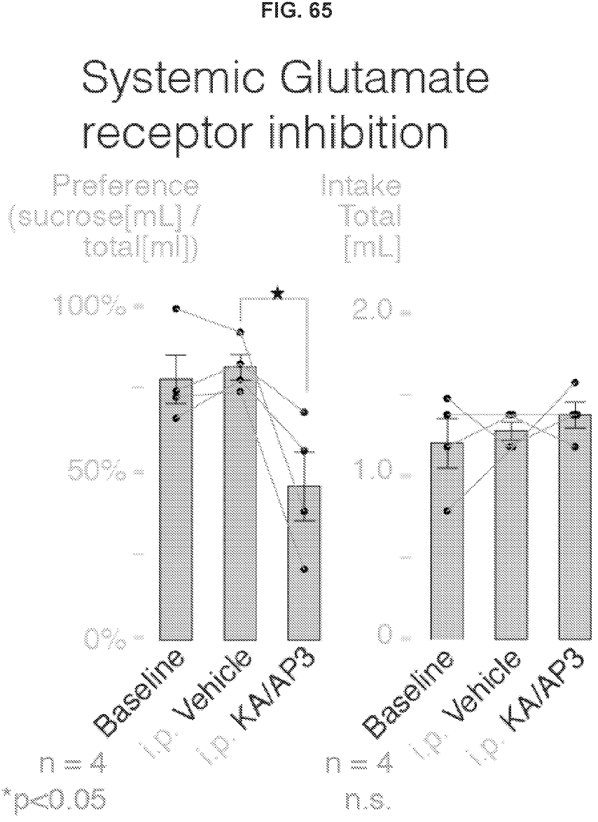

CckCRE_NpH3 mice (n = 8) and littermate controls (n = 5) were implanted with the flexible fiberoptic and given the choice between iso-sweet sucrose [300 mM] and sucralose [15 mM] in a one-hour choice assay (FIG. 38 and FIG. 53). In implanted CckCRE_NpH3 mice, photoinhibition of duodenal neuropod cells eliminated their preference for sucrose (preference for sucrose with silencing 532 nm 58.9% ± 3.92%; with control 473 m 90.8% ± 3.74%; n = 8, p < 0.01; FIG. 53, FIG. 54, and FIG. 55). Under the 1-minute ON 2-minutes OFF inhibition paradigm, the preference-suppressing effect of 532 nm laser was sufficient to supply inhibition throughout the hour and preference suppression extended during the 2-minutes laser OFF (FIG. 56). This suppression of sucrose preference was due to a significant decrease in sucrose consumption and an increase in sucralose consumption (p < 0.05); total consumption of liquid was unchanged (FIG. 55). To ensure laser inhibition was not causing malaise, we found inhibition of neuropod cells with 532 nm laser had no effect on activity during the assay or on 24-hour intake of chow or water compared to 473 nm control conditions (FIG. 57 and FIG. 58). The effect of laser inhibition was specific to mice in which small intestinal neuropod cells expressed the light activated inhibitor channel eNphR3.0 (halorhodopsin). In negative genotype littermate controls, 532 nm or 473 nm light stimulation did not affect the preference for sucrose over sucralose (preference for sucrose with 473 nm 84.2% ± 6.30%, with 532 nm 87.7% ± 2.21 %; n = 5, n.s.; FIG. 59, FIG. 60, and FIG. 61). Whether the caloric neurotransmitter glutamate was contributing to the sucrose preference was tested. Intraperitoneal application of glutamate receptors blockers KA/AP3 [150 µg/kg /1 mg/kg] decreased sucrose preference (n = 4, p < 0.05; FIG. 62, FIG. 63, FIG. 64, and FIG. 65), however, the effect had a delayed onset. Therefore, to reduce the confounding impact of slow diffusion and off-target effects of global delivery of glutamate receptor blockers, a lower dose of KA/AP3 [5 ng/0.1 µg in 0.4 mL] was intraduodenally infused over the 1-hour choice test. lntraduodiodenal inhibition of glutamate receptors eliminated the preference for sucrose (preference for sucrose with vehicle 82.4% ± 3.20%; with KA/AP3 44.0% ± 5.19%; n = 4, p < 0.05; FIG. 66 and FIG. 67). These data show that neuropod cells release glutamate to drive preference for caloric sugar.

While the effects of the cardinal senses on what is eaten have been scrutinized, the gut’s contribution to rapidly sensing and synaptically communicating with the brain has been largely overlooked. McCrickerd and Forde, Obesity Rev. 17(1): 18-29 (2016). The gut, however, represents one of the largest sensory organs. Furness et al., Nat. Rev. Gastroenterol. Hepatol. 10(12): 729-740 (2013). Indeed, signals arising from the small intestine have been shown to drive preference for sugar but uncovering the effects of specific cell types in gut-brain signaling has been limited by a focus on slow, hormonal signaling and by the specificity and rapidity offered by available tools. Thus, the identity of the gut sensor underlying sugar preference has remained unknown, until now. It was demonstrated herein that sugar preference was eliminated by inhibition of gut neuropod cells, an effect recapitulated by local glutamate receptor inhibition. This discovery unveils the possibility of changing behaviors from the gut. This epithelial calorie-sensor cell could be manipulated to alter our perception of what we eat in both health and disease. By differentially transducing the properties of ingested nutrients to the brain, neuropod cells form a gut sense for calories.

### Example 8

### A Gut Sensor for Fatty Acids

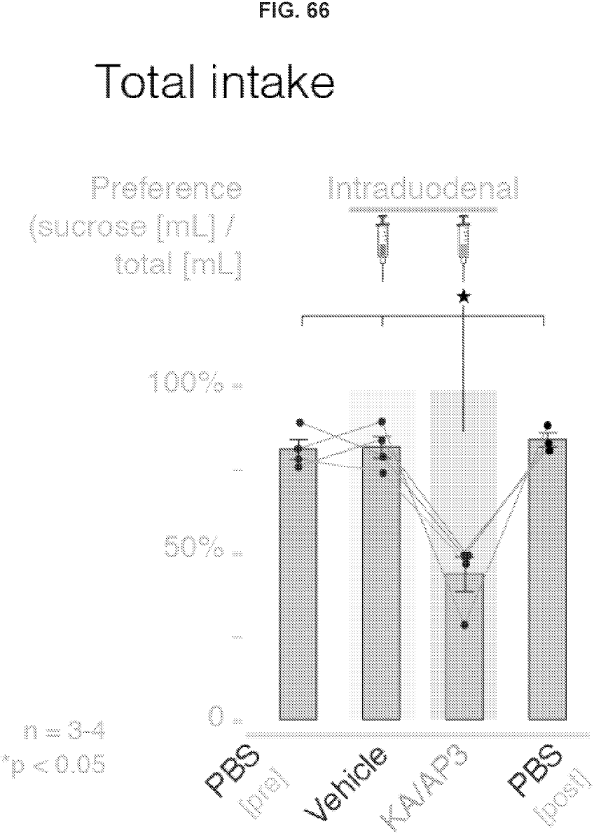

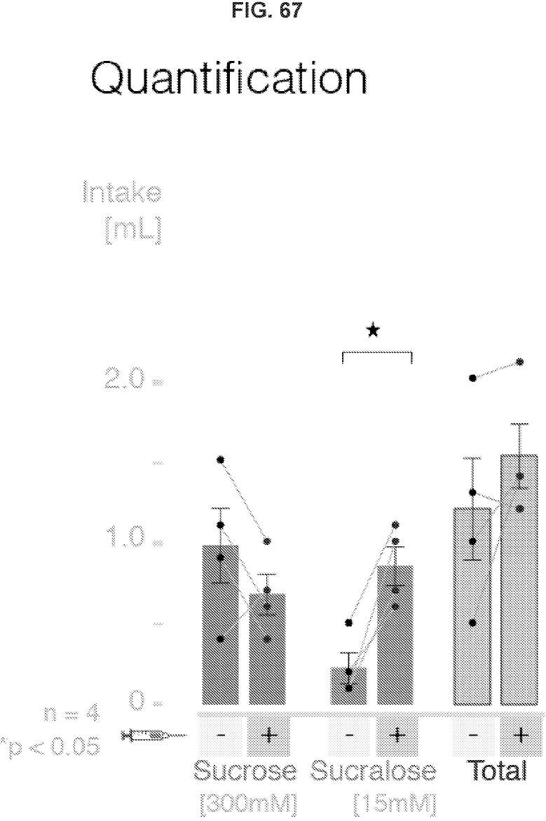

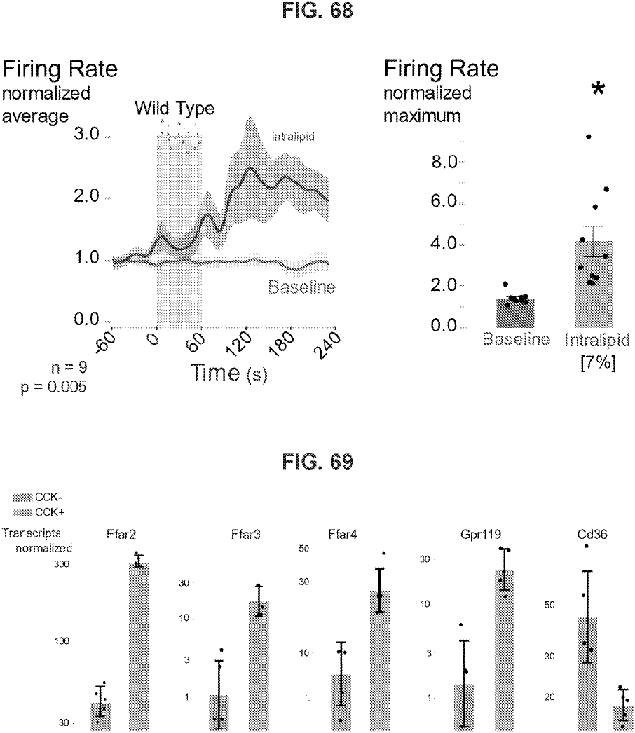

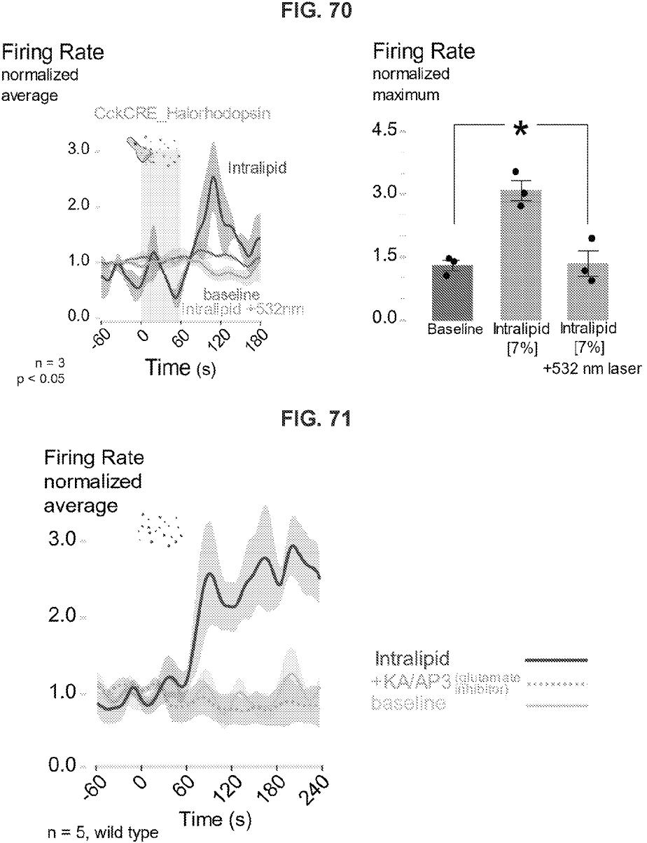

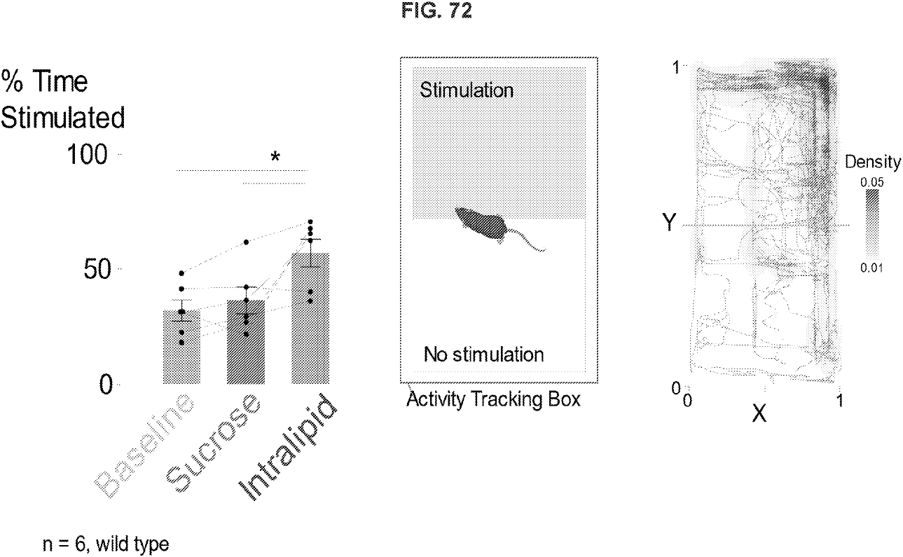

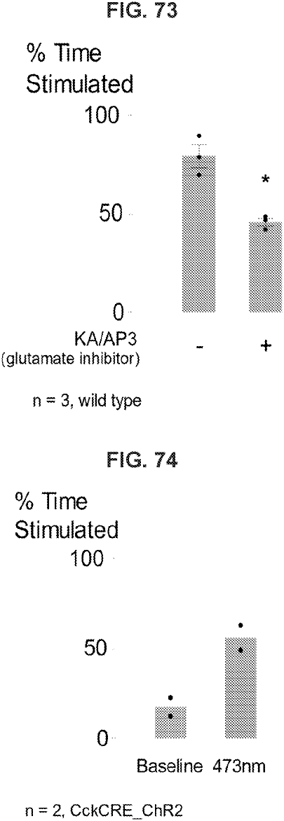

A cocktail of long-chain fatty acids (Intralipid, 7%) significantly increases vagal firing rate when perfused through the duodenum of wild type mice on a C57Bl6ij background (FIG. 68). Cck neuropod cells in the duodenum express receptors for fatty acids (FIG. 69), analyzed by bulk sequencing Cck-positive and Cck-negative epithelial cells in the duodenum of CckGFP mice. Thus, CCK cells in the duodenum may signal the presence of fatty acids to the vagus nerve. To test this, the silencing optogenetic channel was expressed in Cck cells of mice. Using 532 nm light to silence Cck cells in the proximal duodenum, intralipid did not increase vagal firing rate (FIG. 70). In the duodenum, there is overlap in cells expressing CCK and the vesicular glutamate transporter 1 (Vglut1), required for release of the neurotransmitter glutamate. To test whether glutamate is required as a signaling molecule for fat transduction to the vagus nerve, a cocktail of glutamate receptor inhibitors was perfused through the duodenum of wild type mice. Blockade of ionotropic and metabotropic glutamate receptors blocked vagal firing elicited by glutamate (FIG. 71). To test how duodenal fatty acids impact behavior, wild type mice were implanted with chronic, indwelling catheters to the duodenum and tested in a Real Time Place Preference assay. In an initial baseline test, mice freely explore an arena for 20 minutes, during which their position in the arena measured. The initially non-preferred side is deemed the active side. In subsequent tests, the mouse’s position stimulated duodenal perfusion of sucrose [300 mM], known to stimulate vagal firing through Cck cells, or intralipid [4%], equicaloric to 300 mM sucrose. Intralipid, but not sucrose, caused a significant real time place preference (FIG. 72). This indicates that intralipid infused in the duodenum is rewarding. To test whether glutamate is required for intralipid real time place preference, a cocktail of glutamate receptor inhibitors was delivered intraduodenally 20 minutes before the real time place preference test. Glutamate receptor blockade significantly decreased real time place preference for intralipid (FIG. 73). To test whether neuropod cell stimulation is sufficient to elicit a real time place preference, the test was repeated in mice expressing the activating optogenetic channel Channelrhodopsin (ChR2) in Cck cells. These mice were implanted with a flexible PC/PMMA fiberoptic. When mice crossed into the active side, 473 nm light stimulated Cck cells. This activation caused a robust real time place preference (FIG. 74).

What is claimed:

1. A method for modulating a behavior or emotion through a neuroepithelial circuit between a gut sensory cell and the brain, the method comprising stimulating or inhibiting a transsynaptic signal from the gut sensory cell to the brain.

2. A method for modulating a behavior or emotion through a neuroepithelial circuit between a gut sensory cell and the brain, the method comprising stimulating or inhibiting glutamate release from the gut sensory cell.

3. A method for modulating a behavior or emotion through a neuroepithelial circuit between a gut sensory cell and the brain, the method comprising stimulating or inhibiting ATP release from the gut sensory cell.

4. The method of any one of claims 1-3, wherein in the behavior or emotion comprises feeding or consumption, hunger, satiety, appetite, craving, anxiety, depression, addiction, compulsion, pleasure, or combinations thereof.

5. The method of any one of claims 1-4, wherein the transsynaptic signal is stimulated or inhibited by stimulating or inhibiting a receptor on the gut sensory cell.

6. The method of any one of claims 1-5, wherein the receptor comprises a sweet taste receptor, a fatty acid receptor, a sodium glucose like transporter, or a combination thereof.

7. The method of any one of claims 1-6, wherein the sweet taste receptor comprises a T1R2 subunit, a T1R3 subunit, or a combination thereof.

8. The method of any one of claims 1-7, wherein the sodium glucose like transporter is SGLT1.

9. The method of any one of claims 1-8, wherein the fatty acid receptor comprises Ffar2, Ffar3, Ffar4, Gpr119, Gp120, Cd36, or a combination thereof.

10. The method of any one of claims 1-9, wherein a caloric sugar stimulates release of glutamate from the gut sensory cell.

11. The method of any one of claims 1-10, wherein a non-caloric sugar stimulates release of ATP from the gut sensory cell.

12. The method of any one of claims 1-11, wherein a fatty acid stimulates release of glutamate from the gut sensory cell.

13. The method of any one of claims 1-12, wherein a non-caloric fatty acid stimulates release of ATP from the gut sensory cell.

14. The method of any one of claims 1-13, wherein the transsynaptic signal from the gut sensory cell to the brain is conveyed by the vagus nerve that comprises a glutamate receptor, an ATP receptor, or a combination thereof.

15. The method of any one of claims 1-14, wherein stimulation of the glutamate receptor on the vagus nerve induces a preference for a caloric sugar in a subject.

16. The method of any one of claims 1-15, wherein inhibition of the glutamate receptor on the vagus nerve abolishes the preference for a caloric sugar in a subject.

17. The method of any one of claims 1-16, wherein stimulation of the glutamate receptor on the vagus nerve induces a preference for a caloric fatty acid in a subject.

18. The method of any one of claims 1-17, wherein inhibition of the glutamate receptor on the vagus nerve abolishes the preference for a caloric fatty acid in a subject.

19. The method of any one of claims 1-18, wherein stimulating or inhibiting the receptor on the vagus nerve comprises contacting the vagus nerve with a composition capable of stimulating or inhibiting the receptor on the vagus nerve.

20. The method of any one of claims 1-19, wherein the composition comprises a modulator of the receptor.

21. The method of any one of claims 1-20, wherein the modulator of the receptor comprises an agonist of the receptor, an antagonist of the receptor, or a combination thereof.

22. The method of any one of claims 1-21, wherein the modulator of the receptor comprises a glutamate receptor antagonist.

23. The method of any one of claims 1-22, wherein the glutamate receptor antagonist is kynurenic acid, D-/L-2-amino-3-phosphonopropionic acid (AP3), or a combination thereof.

24. The method of any one of claims 1-23, wherein the modulator of the receptor comprises an ATP receptor antagonist.

25. The method of any one of claims 1-24, wherein the ATP receptor antagonist is PPADS.

26. The method of any one of claims 1-25, wherein a subject prefers a caloric sugar to a non-caloric sugar wherein inhibition of the gut sensory cell abolishes the preference.

27. The method of any one of claims 1-26, wherein a subject prefers a caloric fatty acid to a non-caloric fatty acid wherein inhibition of the gut sensory cell abolishes the preference.

28. A method of modulating calorie consumption behavior or emotion through a neuroepithelial circuit between a gut sensory cell and the brain, the method comprising stimulating or inhibiting a transsynaptic signal from the gut sensory cell to the brain.

29. The method of claim 28, wherein the calorie consumption behavior or emotion in a subject is decreased.

30. The method of claim 28 or 29, wherein the calorie consumption behavior or emotion in a subject is increased.

31. The method of any one of claims 28-30, wherein the calorie consumption behavior or emotion comprises feeding or consumption, hunger, satiety, appetite, craving, anxiety, depression, addiction, compulsion, pleasure, or combinations thereof.

32. A method for modulating a neurological or mental health condition comprising stimulating or inhibiting a transsynaptic signal from a gut sensory cell to the brain.

33. The method of claim 32, wherein the neurological or mental health conditions comprise anxiety, depression, autism, eating disorders, memory loss, neurologic pain, alcoholism, drug addiction, compulsive disorders, or combinations thereof.

34. The method of claim 32 or 33, wherein symptoms of the neurological or mental health condition are reduced.

35. The method of any one of claims 32-34, wherein stimulating or inhibiting the receptor on the gut sensory cell comprises administering to a subject a therapeutically effective amount of a composition capable of stimulating or inhibiting the receptor on the gut sensory cell of the subject.

36. The method of any one of claims 32-35, wherein stimulating or inhibiting the receptor on the gut sensory cell comprises contacting the gut sensory cell with a composition capable of stimulating or inhibiting the receptor on the gut sensory cell.

37. The method of any one of claims 32-36, wherein the composition comprises a modulator of the receptor.

38. The method of any one of claims 32-37, wherein the modulator of the receptor comprises an agonist of the receptor, an antagonist of the receptor, or a combination thereof.

39. The method of any one of claims 32-38, wherein the modulator of the receptor comprises an SGLT1 antagonist.

40. A method for treating or ameliorating obesity comprising stimulating or inhibiting a transsynaptic signal from a gut sensory cell to the brain.

41. A method for distinguishing caloric from non-caloric sugars, the method comprising stimulating or inhibiting receptors on a gut sensory cell that modulates a transsynaptic signal from the gut sensory cell to the brain.

42. The method of claim 40 or 41, wherein the receptors comprise a sweet taste receptor, a sodium glucose like transporter, or a combination thereof.

43. The method of any one of claims 40-42, wherein the sweet taste receptor comprises a T1R2 subunit, a T1R3 subunit, or a combination thereof.

44. The method of any one of claims 40-43, wherein the sodium glucose like transporter is SGLT1.

45. The method of any one of claims 40-44, wherein the caloric sugar comprises glucose, sucrose, maltodextrin, dextrose, maltose, fructose, galactose, or a combination thereof.

46. The method of any one of claims 40-45, wherein the non-caloric sugar comprises sucralose, aspartame, saccharin, acesulfame-K, neotame, stevia, or a combination thereof.

47. The method of any one of claims 40-46, wherein the composition comprises between about 0.5 mM and about 1000 mM of a caloric or non-caloric sugar.

48. The method of any one of claims 40-47, wherein the composition comprises between about 2 mM and about 500 mM of a caloric or non-caloric sugar.

49. The method of any one of claims 40-48, wherein the composition comprises at least about 100 mM a caloric or non-caloric sugar.

50. The method of any one of claims 40-49, wherein the composition comprises at least about 300 mM sucrose.

51. The method of any one of claims 40-50, wherein the composition comprises at least about 2 mM of sucralose.

52. The method of any one of claims 40-51, wherein the transsynaptic signal from the gut sensory cell to the brain comprises a neuroepithelial circuit that comprises a nerve fiber.

53. The method of any one of claims 40-52, wherein the neuroepithelial circuit comprises a gut sensory cell in contact with the nerve fiber.

54. The method of any one of claims 40-53, wherein the gut sensory cell is in contact with the nerve fiber by releasing a neurotransmitter.

55. The method of any one of claims 40-54, wherein the neurotransmitter is glutamate.

56. The method of any one of claims 40-55, wherein the nerve fiber is a vagal nerve fiber or a sensory nerve fiber.

57. The method of any one of claims 40-56, wherein the vagal nerve fiber includes a vagal nodose neuron.

58. The method of any one of claims 40-57, wherein the gut sensory cell comprises a gut epithelial cell.

59. The method of any one of claims 40-58, wherein the gut sensory cell comprises an enteroendocrine cell and/or an enterochromaffin cell.

60. The method of any one of claims 40-59, wherein the neuroepithelial circuit comprises an enteroendocrine cell in contact with a vagal nerve fiber.

61. The method of any one of claims 40-60, wherein the subject is a human subject.

62. The method of any one of claims 40-61, wherein the receptor on the nerve fiber is an ionotropic glutamate receptor.

63. The method of any one of claims 40-62, wherein the receptor on the nerve fiber is an N-methyl-D-aspartate (NMDA) receptor, an α-amino-3-hydroxy-5-methyl-4-isoxazolepropionic acid (AMPA) receptor, or a kainate receptor.

64. A method for distinguishing caloric from non-caloric fatty acids, the method comprising stimulating or inhibiting receptors on a gut sensory cell that modulates a transsynaptic signal from the gut sensory cell to the brain.

65. The method of claim 64, wherein the receptors comprise a fatty acid receptor.

66. The method of claim 64 or 65, wherein the fatty acid receptor comprises Ffar2, Ffar3, Ffar4, Gpr119, Gp120, Cd36, or a combination thereof.
